SEQUENCE LISTING (2 sequences)

SEQ ID NO: 1 - DNA, 9680 bp, Artificial Sequence
  HIV delta A3Vif+
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattacggaaaacagatggcaggtgacgattgtgtggcaagtagacaggacgaggattaacacatggaaaagattagtaaaacaccattaatatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagttaaaggtgtgaatatcaagcaggacataacaaggtaggatctctacagtacttggcactagcagcattaataaaaccaaaacagataaagccacctttgcctagtgttaggaaactgacagaggacagcccgaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaataaataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaccccaacctataatagtagcaatagtagcattagtagtagcaataataatagcaatagttgtgtggtccatagtaatcatagaatataggaaaatattaagacaaagaaaaatagactaattaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagcccggtggcaagtggtcaaaaagtagtgtgattggatggcctgctgtaagggaaagataaagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaataatacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacc

SEQ ID NO: 2 - DNA, 9680 bp, Artificial Sequence; identical to SEQ ID NO: 1
  HIV delta A4

(19) United States
(12) Patent Application Publication
(10) Pub. No.: US 2023/0279388 A1
(43) Pub. Date: Sep. 7, 2023

(54) Replication Competent Virus Assay

(71) Applicant: Oxford BioMedica (UK) Limited, Oxford (GB)

(72) Inventors: Samuel James Stockdale, Oxford (GB); Rui Andre Saraiva Raposo, Oxford (GB); Daniel Farley, Oxford (GB); Nicholas George Clarkson, Oxford (GB)

(21) Appl. No.: 17/909,427

(22) PCT Filed: Mar. 8, 2021
(86) PCT No.: PCT/GB2021/050570

§ 371 Date: Sep. 5, 2022

(30) Foreign Application Priority Data
Mar. 9, 2020 (GB) 2003412.0

(51) Int. Cl. C12N 15/10

(52) U.S. Cl. CPC C12N 15/1082

(57) ABSTRACT

The present invention provides a novel method for detecting replication competent virus in a test sample. The method comprises culturing and diluting a plurality of individual cell culture aliquots comprising virus-permissive cells and a portion of the test sample, followed by testing for the presence of replication competent virus. The method may be used in parallel with a positive control, which is also provided herein.

The present invention provides a novel method for detecting replication competent virus in a test sample. The method comprises culturing and diluting a plurality of individual cell culture aliquots comprising virus-permissive cells and a portion of the test sample, followed by testing for the presence of replication competent virus. The method may be used in parallel with a positive control, which is also provided herein.

### BACKGROUND

Gene therapy broadly involves the use of genetic material to treat disease. Therapeutic genetic material may be incorporated into the target cells of a host using vectors to enable the transfer of nucleic acids. Such vectors can generally be divided into viral and non-viral categories. The use of viral vectors for delivery of therapeutic genes is well known and gene therapy products are now an important part of our global healthcare markets.

Viruses naturally introduce their genetic material into target cells of a host as part of their replication cycle. Engineered viral vectors harness this ability to enable the delivery of a nucleotide of interest (NOI) to a target cell. To date, a number of viruses have been engineered as vectors for gene therapy. These include retroviruses, adenoviruses (AdV), adeno-associated viruses (AAV), herpes simplex viruses (HSV) and vaccinia viruses (VACV).

Retroviral vectors have been developed as therapies for various genetic disorders and continue to show increasing promise in clinical trials and approved therapeutic products (e.g. Strimvelis™ and KymriahTM, amongst others). Currently there are over 459 human clinical trials involving retroviral gene therapy registered in the Journal of Gene Medicine database; 158 gene therapy clinical trials are using lentiviral vectors (http://www.abedia.com/wiley/vectors.php, updated in April, 2017).

Viral vectors for use in gene therapy are typically engineered to be replication defective. As such, the recombinant vectors can directly infect a target cell, but are incapable of producing further generations of infective virions. Other types of viral vectors may be conditionally replication competent within cancer cells only, and may additionally encode a toxic transgene or pro-enzyme.

The manufacture of viral vectors for human gene therapy and vaccination is well documented. Well known methods of viral vector manufacture include transfection of primary cells or mammalian/insect cell lines with vector DNA components, followed by a limited incubation period and then harvest of crude vector from culture media (referred to as “harvest supernatant” herein) and/or cells. Often, each component required for vector production is encoded by separate plasmids, partly for safety reasons, as it would then require a number of recombination events to occur for a replication competent virus particle to be formed through the production process.

Although viral vectors are engineered to be replication defective, in many instances it may be desirable or even necessary to verify the absence of replication competent virus (e.g., as replication competent retrovirus (RCR) or replication competent lentivirus (RCL)) in a sample or composition, such as a therapeutic or pharmaceutical composition formulated for administration. Various methods are available for verifying the absence of replication competent virus, including [1] PCR assays that detect the transcription of genes that are expressed in a retrovirus (and putative RCR/RCL) but not in the viral vector particle (see e.g. WO2019/152747), [2] assays that measure a necessary functional property of a putative RCR/RCL (Sastry et al., 2005), and [3] phenotypic assays such as plaque-forming assays (Forestell et al., 1996). In the majority of assay formats, a cell-based phase is required to amplify any potential RCR/RCL present within the test article to increase confidence/sensitivity, which is then followed by an end-point assay. In cases where the test article (e.g. vector product) shares the same properties (e.g. reverse transcriptase activity) as the putative RCR/RCL, the amplification phase also provides the necessary time to dilute-out this activity such that potential signal from an actual RCR/RCL that may be present can be unambiguously detected by the end-point assay. This is modelled by a suitable positive control virus spiked into amplification cell cultures inoculated with the test article. For RCR/RCL testing the test article is the vector material and also post-production cells, therefore requiring two tests for any given batch of vector product. However, a number of factors complicate the design of replication competent virus (RCV) testing, especially regarding the scale of the culturing phase. RCV arising from viral vector systems being developed for clinical use have been reported in the past, however RCL derived from 3^{rd} generation lentiviral systems have yet to be reported. Therefore, the RCV detection system must anticipate a currently theoretical virus. Accordingly, fifteen or more flasks with at least 40 ml culture each are typically required to initiate each assay, which goes through several passages. Typically, therefore, more than 100 flasks need to be processed over the three to four week time course of the assay. These methods can therefore be time consuming and labour intensive, especially as these testing methods are often required to be carried out at containment level 3, depending on the positive control virus employed. The manual passaging of large volumes in many culture flasks over extended periods of time also increases the likelihood of human error and/or introduction of microbial contamination, both resulting in costly assay failure or termination.

There is a need for an improved method for detecting replication competent virus in a test sample.

### BRIEF SUMMARY OF THE DISCLOSURE

The invention is based on the surprising finding that replication competent virus can still be accurately detected when low individual aliquot volumes (e.g. of less than 12 ml) are used. This finding allows such assays to be more easily automated, for example by using tissue culture plates comprising a plurality of wells that can be processed robotically. Automation of such assays significantly reduces operator workload and increases assay throughput. In addition, the inventors have found that dilution factors of at least 2 can be used during passaging of the low volume aliquots, without adversely affecting sensitivity. Advantageously, the methods described herein may be used to reduce the overall volumes and/or aliquot number used for replication competent virus detection, whilst retaining sensitivity.

The inventors have also investigated which initial cell seeding densities can be used with the unit volumes described herein. Advantageously, they have found that seeding densities of least 1 x 10⁵ total cells/ml can be used. Surprisingly, they have also found that increasing the initial seeding density increases the sensitivity of the assay without having an inhibitory effect on the infection rate of the corresponding positive control. Advantageously, an initial seeding density in the range of from about 1 x 10⁵ total cells/ml to 1 x 10⁷ total cells/ml (e.g. in the range of from about 5 x 10⁵ total cells/ml to about 1 x 10⁷ total cells/ml, such as 1 x 10⁶ total cells/ml to about 1 x 10⁷ total cells/ml) can therefore be used. The seeding densities described herein may therefore be used to increase the rate of infection and/or reduce the total initial volume of test sample required, whilst still adhering to the FDA guidelines.

The methods described herein are useful when manufactured viral products for gene therapy need to be tested before clinical release. In this context, the methods described herein may be performed in parallel with any appropriate positive control (e.g. an attenuated replication competent lentivirus that has at least one accessory gene functionally mutated within its nucleotide sequence, wherein the at least one accessory gene is selected from: vif, vpr, vpx, vpu and nef, as described elsewhere herein). In this context, inventors have also generated a novel vif+, Δvpr, Δvpu and Δnef HIV-1 replication competent virus (referred to as “HIVΔA3Vif+” herein) that is particularly useful when used in combination with the methods described herein, as it maintains infectivity throughout the time course of the assay. This positive control can therefore advantageously be used in the context of the assays described herein.

The inventors have demonstrated the invention using a test sample comprising end of production cells (EOPCs) that were used to manufacture a lentiviral vector. However, the methodology described herein applies equally to methods for detecting replication competent virus in a test sample comprising the manufactured lentiviral vector itself (e.g. harvest supernatant), as the cell culture methods used for testing lentiviral vector or EOPCs relies on the same factors, namely, infectivity, sensitivity and culture in the presence of virus-permissive cells for at least fifteen days (i.e. over the time course of the assay).

The data presented below shows that the novel methods described herein can be used to detect replication competent lentivirus. However, the invention is not limited to lentiviral systems and can be used to detect any replication competent virus such as a retrovirus, adenovirus, adeno-associated virus, herpes simplex virus or vaccinia virus, provided that compatible virus-permissive cells and corresponding positive controls are used. As will be described in more detail below, a person of skill in the art can readily identify compatible virus-permissive cells and corresponding positive controls for use with their virus of choice.

A method for detecting replication competent virus in a test sample is provided comprising:
a) providing a plurality of individual cell culture aliquots each with maximum aqueous volume of less than 12 ml, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus.

Suitably, the virus may be selected from the group consisting of: a retrovirus, an adenovirus, an adeno-associated virus, a herpes simplex virus and a vaccinia virus.

Suitably, the retrovirus may be a lentivirus.

Suitably, the maximum aqueous volume of each individual cell culture aliquot in step a) may be selected from: 11 ml, 10 ml, 5 ml or 3 ml.

Suitably, the total volume across all aliquots may be reduced by at least 50% during step c). Suitably, the test sample may comprise viral particles or end of production cells.

Suitably, the virus-permissive cells may be non-adherent.

Suitably, the virus-permissive cells may be selected from:
a) immortalised T cell lines, optionally wherein the cells are selected from Jurkat, CEM-SS, PM1, Molt4, Molt4.8, SupT1, MT4 or C8166 cells; or
b) non-T cell lines, optionally wherein the cells are selected from HEK293 or 92BR cells.

Suitably, the total volume of the plurality of individual cell culture aliquots of step a) may be at least about 115 ml.

Suitably, the initial seeding density of the plurality of individual cell culture aliquots in step a) may be in the range of from about 1 x 10⁵ total cells/ml to about 1 × 10⁷ total cells/ml.

Suitably, the initial seeding density of the plurality of individual cell culture aliquots in step a) may be in the range of from about 1 × 10⁶ total cells/ml to 1 × 10⁷ total cells/ml.

Suitably, step c) may comprise culturing the aliquots for at least a further eight or nine days.

Suitably, each individual cell culture aliquot may be within a cell culture vessel.

Suitably, the cell culture vessel may be selected from a cell culture tube, a cell culture dish or a cell culture plate comprising a plurality of wells.

Suitably, the cell culture plate comprising a plurality of wells may be selected from the group consisting of: a 4- well, 6- well, 8- well, 12- well, 24- well, 48- well, 96- well and a 384- well cell culture plate.

Suitably, the cell culture plate comprising a plurality of wells may be a 12- well plate or a 24-well plate.

Suitably, the method may be automated.

Suitably, the presence of replication competent virus may be tested using PCR or ELISA.

Suitably, the presence of replication competent virus may be tested using a reverse transcriptase assay.

Suitably, the method may be for detecting replication competent lentivirus in the test sample, and the method may be performed in parallel with a positive control sample comprising an attenuated replication competent lentivirus that has at least one accessory gene functionally mutated within its nucleotide sequence, wherein the at least one accessory gene is selected from: vif, vpr, vpx, vpu and nef.

Suitably, the method may be for detecting replication competent HIV, SIV, SHIV in the test sample, or a variant thereof.

Suitably, the attenuated replication competent lentivirus may have at least three of vif, vpr, vpx, vpu and nef functionally mutated.

Suitably, the attenuated replication competent virus may comprise a nucleic acid sequence according to SEQ ID NO: 1.

Suitably, the method may be for testing products for gene therapy.

Suitably, the method for detecting replication competent virus in a test sample may comprise:
a) providing a plurality of individual cell culture aliquots each with maximum aqueous volume of 10 ml or less, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus. In this example, the total volume of the plurality of individual cell culture aliquots of step a) may be at least about 115 ml.

Suitably, the method for detecting replication competent virus in a test sample may comprise:
a) providing a plurality of individual cell culture aliquots each with maximum aqueous volume of 5 ml or less, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus. In this example, the total volume of the plurality of individual cell culture aliquots of step a) may be at least about 115 ml.

Suitably, the method for detecting replication competent virus in a test sample may comprise:
a) providing a plurality of individual cell culture aliquots each with maximum aqueous volume of 3 ml or less, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus. In this example, the total volume of the plurality of individual cell culture aliquots of step a) may be at least about 115 ml.

Suitably, the method for detecting replication competent virus in a test sample may comprise:
a) providing a plurality of individual cell culture aliquots each with maximum aqueous volume of 2 ml or less, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus. In this example, the total volume of the plurality of individual cell culture aliquots of step a) may be at least about 115 ml.

Suitably, the method for detecting replication competent virus in a test sample may comprise:
a) providing a plurality of individual cell culture aliquots each with maximum aqueous volume of 1 ml or less, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus. In this example, the total volume of the plurality of individual cell culture aliquots of step a) may be at least about 115 ml.

Suitably, the method for detecting replication competent virus in a test sample may comprise:
a) providing a plurality of individual cell culture aliquots each with maximum aqueous volume of 0.6 ml or less, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus. In this example, the total volume of the plurality of individual cell culture aliquots of step a) may be at least about 115 ml.

A replication competent virus comprising a nucleic acid sequence according to SEQ ID NO: 1 is also provided.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows the effect of increasing initial seeding density on calculated infectious titre (Operator 1).

FIG. 2 shows the effect of increasing initial seeding density on calculated infectious titre (Operator 2).

FIG. 3 shows a schematic showing a particular example in which 8× 24-well plates are sequentially pooled to 1× 12-well plate over the duration of the assay.

FIG. 4 shows a schematic showing the accessory gene knock-outs used to generate an RCL assay positive control virus. Wild type (wt) HIV-1 provirus genome structure is displayed; the U3 promoter drives transcription, which is activated by tat. Unspliced and single spliced mRNA encodes for gagpol and env proteins respectively, and the unspliced vRNA is packaged into virions. The unspliced mRNA requires rev in trans to be exported from the nucleus. The accessory genes vif, vpr, vpu and nef are absent from the vector system, and generally are only required for replication in primary cells. A suitable positive control virus for RCL assays ideally should be the most attenuated version of the parental virus from which the vector system is based (in this case HIV-1). The attenuated variants HIVΔA3Vif+ and HIVΔA4 encode/express tat and rev, which are absolutely required for replication. The accessory genes have been functionally mutated in both variants, except for the requirement of Vif in HIVΔA3Vif+ when using C8166-45 cells because these cells express low levels of the restriction factor APOBEC3G, which Vif counteracts.

FIG. 5 shows an overview and data from an experiment performed demonstrating that fully attenuated HIV-1 (HIVΔA4) loses infectivity during passage in C8166 cells. HIVΔA4 proviral DNA harbouring functional mutations in vif, vpr, vpu and nef was first produced in HEK293T cells by transient transfection. The resulting HIVΔA4 virus stock was titrated by F-PERT (RT-qPCR) to quantify the number of RT units. Then the virus stock was titrated on C8166 cells, infecting with 10-fold serially diluted virus in triplicate (1000-to-1 RT unit per well) and then de novo production of HIVΔA4 was measured after several days post-inoculation by F-PERT. The positive-negative threshold for infection was set at Ct 25 in the F-PERT assay; below Ct 25 indicated unambiguous de novo generation of HIVΔA4 prior to passage 1 (Passage 0). In parallel, a main culture of C8166 cells was inoculated with 100 RT-units the HIVΔA4 virus stock and passaged six times before generating a virus PC bank intended for use in RCL assays. However, upon repeating the F-PERT analysis of this final HIVΔA4 virus stock as performed at passage 0, it was shown that despite inoculating fresh C8166 cells with RT-unit matched amounts of HIVΔA4 virus, the production of de novo HIVΔA4 was dramatically reduced compared to the starting virus stock. This indicated that the virus passaged in C8166 cells had become attenuated over the time-course. It was hypothesised that this was due to low level expression of APOBEC3G in C8166 cells reported in the literature.

FIG. 6 shows ethidium-bromide stained agarose gel analysis of restriction enzyme digest of plasmid DNA encoding HIVΔA4 or HIVΔA3Vif+, demonstrating successful ‘re-insertion’ of the Vif ORF by cloning.

FIG. 7 shows an overview and data from an experiment performed demonstrating that Vif function is required to maintain virus activity through long term infection of C8166 cells. Wild type HIV-1 (NL4-3), HIVΔA4 and HIVΔA3Vif+ virus stocks were first produced in HEK293T cells by transient transfection. In T25 flasks 1.5×10⁶ C8166 cells were infected with 100 RT units of each virus stock, and cultured for 3-4 days to allow de novo virus production. At each passage point, cell-free culture supernatant was sampled and analysed for RT activity (F-PERT assay), before fresh C8166 cell cultures were inoculated with 0.1 mL cell-free supernatant (i.e. supernatant-only passaging of virus). The RT activity in supernatant at each passage point was plotted, indicating that HIVΔA4 gradually lost the ability to infect new cells, whereas the HIVΔA3Vif+ virus was able to productively infect cells leading to maximally infected cultures at each point prior to passaging. Sequencing of a region within Pol in the three virus genomes at passage 6, revealed ~10 times more G>A hypermutation events within HIVΔA4 compared to wild type or HIVΔA3Vif (data not shown), in-line with the premise that loss of infectivity by HIVΔA4 is due to semi-restrictive levels of APOBEC3G.

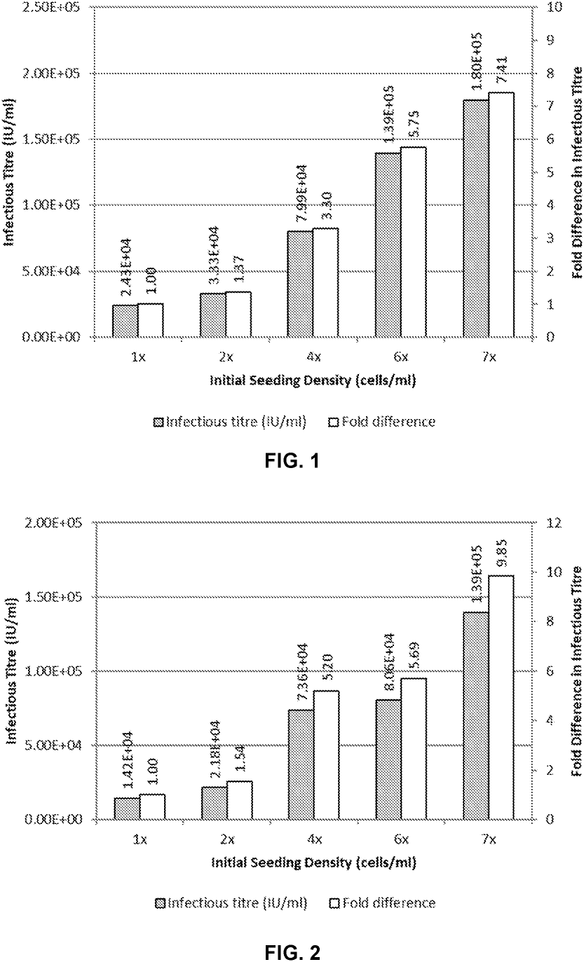

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

### Methods for Detecting Replication Competent Virus

A novel method for detecting replication competent virus in a test sample is provided herein. The method comprises culturing and diluting a plurality of individual cell culture aliquots comprising virus-permissive cells and a portion of the test sample, followed by testing for the presence of replication competent virus. The methods described herein are particularly useful for testing products for gene therapy.

As used herein, a “test sample” refers to any sample of interest that may comprise a replication competent virus. Typically, whether or not the test sample comprises a replication competent virus is unknown at the start of the method.

In a particular example, the test sample comprises viral particles or end of production cells.

Accordingly, in one example, the test sample may comprise viral particles. Viral particles are also referred to as viral vector particles, virions or viruses herein. The viral particles may be present within a cell culture supernatant that is harvested during the manufacture of viral vector for gene therapy. Such cell culture supernatants are also referred to herein as a harvest supernatant. The test sample may therefore be a harvest supernatant. Typically, such assays are referred to as “replication competent virus” assays (RCV assays, e.g. RCR assays (for retrovirus) or RCL assays (for lentivirus)).

In another example, the test sample may be an end of production cell sample. Typically, such assays are referred to as “replication competent virus co-culture” assays (RCVCC assays, e.g. RCRCC assays (for retrovirus) or RCLCC assays (for lentivirus)) because they require co-culture of the end of production cells with virus-permissive cells. The term “end of production cell sample” refers to any sample that comprises end of production cells. “End of production cells” are cells that have been used for manufacture of a viral vector. In other words, they are the production cells that remain at the end of the manufacturing cycle. Production cells are also known as viral vector production cells, or vector production cells.

A “viral vector production cell”, “vector production cell”, or “production cell” is to be understood as a cell that is capable of producing a viral vector or viral vector particle. Vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of viral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. They may be cells cultured in vitro such as a tissue culture cell line. They are generally mammalian cells but can be, for example, insect cells. Suitable mammalian cells include murine fibroblast derived cell lines or human cell lines. Preferably the vector production cells are derived from a human cell line. Non-limiting examples of suitable eukaryotic cells such as mammalian or human cells, include HEK293T, HEK293, CAP, CAP-T, CHO cells, or PER.C6 cells. A non-limiting example of a suitable insect cell may be SF9 cells.

Methods for introducing nucleic acids into production cells are well known in the art and have been described previously.

The methods described herein are for detecting the presence of replication competent virus in the test sample. As used herein, “replication competent virus” refers to a virus that is able to replicate, i.e. it is not, or is no longer, replication deficient. As such, the virus can directly infect a target cell and is capable of producing further generations of infective virions.

Any appropriate virus may be detected using the methods described herein. For example, the virus may be able to infect a mammalian (preferably human) cell. Appropriate viruses may be selected from the group consisting of: a retrovirus, an adenovirus, an adeno-associated virus, a herpes simplex virus and a vaccinia virus. For example, the virus may be a lentivirus. In one example, the virus is a SIN (self-inactivating) virus. In some examples, the virus of interest may be selected MMLV, HIV-1, EIAV or variants thereof. Details of each of these viruses is provided in the “general definitions” section below.

### Providing a Plurality of Individual Cell Culture Aliquots

The methods described herein comprise the step of a) providing a plurality of individual cell culture aliquots each with maximum aqueous volume of less than 12 ml (e.g. a maximum aqueous volume of 11 ml, 10 ml, 5 ml or 3 ml as appropriate), wherein each aliquot comprises a portion of the test sample and virus-permissive cells. By using a plurality of aliquots with relatively small volumes, the methods provided herein can more easily be automated. It is surprising that, in the context of RCR and RCRCC assays (and their equivalents, including RCL and RCLCC assays), using a plurality of aliquots with small culture volumes retains sensitivity over the assay. Sensitivity is crucial in this context as such detection systems must anticipate a currently theoretical virus.

The methods described herein are particularly advantageous when used in combination with a plurality of individual cell culture aliquots each with a small maximum aqueous volume (e.g. of 3 ml or less), because such volumes are particularly relevant for automation.

As used herein, a “individual cell culture aliquot” (also abbreviated to “aliquot” herein) refers to a discrete cell culture volume that is present within a single cell culture reaction chamber. In other words, it refers to the total amount of cell culture composition that is present within an individual cell culture reaction chamber. The cell culture reaction chamber may be a cell culture well (e.g. a well within a cell culture plate), a cell culture tube, a cell culture dish or a cell culture flask.

Cell culture tubes, cell culture flasks, cell culture dishes and cell culture plates are referred to herein as cell culture vessels as they are examples of discrete cell culture products (or consumables) that may be used within the methods described herein. Cell culture tubes, cell culture flasks, cell culture dishes are typically cell culture vessels with a single cell culture reaction chamber, whereas cell culture plates are typically cell culture vessels with several cell culture reaction chambers (i.e. several wells). Other appropriate cell culture vessels are well known in the art.

As a specific example, therefore, a cell culture vessel may be a cell culture plate comprising a plurality of wells. In this context, the cell culture vessel (plate) has several cell culture reaction chambers (wells), which are each capable of holding an individual cell culture aliquot (discrete volume of cell culture).

Optionally, the cell culture vessel is selected from a cell culture tube, a cell culture dish or a cell culture plate comprising a plurality of wells. Preferably, the cell culture vessel is a cell culture plate comprising a plurality of wells, as this format is most suitable for automation. For example, the cell culture plate may be selected from a 4- well, 6- well, 8- well, 12- well, 24- well, 48- well, 96- well or 384- well cell culture plate. In a particular example, 12- well plates and/or 24- well plates may be used. As would be clear to a person of skill in the art, in the context of a cell culture plate comprising a plurality of wells, each well is considered to be a separate cell culture reaction chamber that may contain an individual cell culture aliquot. Accordingly, a 4-well plate is a cell culture vessel that may comprise up to four individual cell culture aliquots (one in each of its separate cell culture reaction chambers/wells); a 6-well plate is a cell culture vessel that may comprise up to six individual cell culture aliquots (one in each of its separate cell culture reaction chambers/wells) etc.

In one example, the individual cell culture aliquots are present within a cell culture vessel that is a cell culture plate, as cell culture plates are particularly amenable to automation and may be used in high throughput assays. As would be clear to a person of skill in the art, under certain circumstances, it may also be useful to use a cell culture tube, as such cell culture vessels may also be used in automated methods (e.g. strips of Eppendorf tubes may be used). Cell culture dishes may also be used in automated methods. Accordingly, in some examples, the individual cell culture aliquots may be present within a cell culture vessel that is a cell culture plate, dish or tube. In some examples, the cell culture vessel is not a cell culture flask.

In a preferred example, the plurality of individual cell culture aliquots are in one (or more) cell culture plate(s). The plurality of individual cell culture aliquots (e.g. each with a small maximum aqueous volume (e.g. of less than 12 ml, e.g. of 3 ml or less), may therefore be present within one or more cell culture plates, where the wells of the plate(s) contain the aliquots (one aliquot per well). In this context, for example, 48 or more individual cell culture aliquots may be provided within two or more 24-well cell culture plates (i.e. with each aliquot being provided within a separate well within the plates). Other examples of how the plurality of aliquots may be provided (e.g. in the format of one or more 4- well, 6- well, 8- well, 12-well, 24- well, 48- well, 96- well or 384- well cell culture plate, or combinations thereof) may be readily identified by a person of skill in the art.

As used herein, “plurality of individual cell culture aliquots” refers to two or more individual cell culture aliquots. The methods described herein may provide 8 or more, 16 or more, 24 or more, 32 or more, 40 or more, 48 or more, 56 or more, 64 or more, 72 or more, 80 or more, 88 or more, 96 or more, 104 or more, 112 or more, 120 or more, 128 or more, 136 or more, 144 or more, 152 or more, 160 or more, 168 or more, 176 or more, 184 or more, 192 or more, 384 or more etc individual cell culture aliquots in step a).

The method provided herein encompasses situations wherein the plurality of individual cell culture aliquots are cultured in parallel (i.e. simultaneously) as well as situations wherein the plurality of individual cell culture aliquots are cultured sequentially (i.e. not at exactly the same time). For example, the method encompasses situations wherein the individual cell culture aliquots are cultured in batches of e.g. 8, 12, 24, 48, 96 etc, wherein each batch is cultured sequentially until a total batch of e.g. 192 individual cell culture aliquots have been cultured and are ready for testing for the presence of replication competent virus. However, in general, simultaneous culture is preferred.

In a particular example, 48 or more individual cell culture aliquots are provided in step a). In another example, 96 or more individual cell culture aliquots are provided in step a). In another example, 120 or more individual cell culture aliquots are provided in step a). In a further example, 192 or more individual cell culture aliquots are provided in step a). in another example, 384 or more individual cell culture aliquots are provided in step a).

As stated herein, each individual cell culture aliquot of step a) has a maximum aqueous volume of less than 12 ml.

The plurality of individual cell culture aliquots of step a) may all have the same volume, or may have varying volumes, provided that the maximum aqueous volume of each individual cell culture aliquot is less than 12 ml. As used herein, a “maximum aqueous volume” refers to the total aqueous volume that can be used for the individual cell culture aliquot. In other words, the aqueous volume of the individual cell culture aliquots may be less than 12 ml (e.g. 11 ml, 10 ml, 5 ml, or 3 ml, or less).

As would be clear to a person of skill in the art, an “individual cell culture aliquot” must have a volume, or it would not be an aliquot. The minimum aqueous volume of an aliquot cannot therefore be zero. A reasonable lower limit to the minimum aqueous volume will depend on the reaction chamber used. For example, a lower limit may be set at 0.1 ml. In other words, the aqueous volume of the individual cell culture aliquots may be less than 12 ml (e.g. 11 ml, 10 ml, 5 ml, or 3 ml, or less etc), with a minimum aqueous volume of 0.1 ml. The individual cell culture aliquots described herein may therefore be considered as having an aqueous volume in the range of from about 0.1 ml to the desired maximum aqueous volume (less than 12 ml, 11 ml, 10 ml, 5 ml, 3 ml etc).

As stated above, the methods described herein are particularly advantageous when used in combination with aliquots with a maximum aqueous volumes of 3 ml because such volumes are conventionally used in automated methods. Accordingly, the aqueous volume of the individual cell culture aliquots may preferably be 3 ml, or less than 3 ml (e.g. 2.9 ml or less, 2.8 ml or less, 2.7 ml or less, 2.6 ml or less, 2.5 ml or less, 2.4 ml or less, 2.3 ml or less, 2.2 ml or less, 2.1 ml or less 2.0 ml or less, 1.9 ml or less, 1.8 ml or less, 1.7 ml or less, 1.6 ml or less, 1.5 ml or less, 1.4 ml or less, 1.3 ml or less, 1.2 ml or less, 1.1 ml or less, 1.0 ml or less, 0.9 ml or less. 0.8 ml or less, 0.7 ml or less, 0.6 ml or less, 0.5 ml or less, 0.4 ml or less, 0.3 ml or less, 0.2 ml or less etc). Typically, as stated above, the minimum aqueous volume of each aliquot may be 0.1 ml.

In a particular example, the aqueous volume of the individual cell culture aliquots may be 2 ml or less in step a). In another example, the aqueous volume of the individual cell culture aliquots may be 1 ml or less in step a). In a further example, the aqueous volume of the individual cell culture aliquots may be 0.6 ml or less in step a).

As would be clear to a person of skill in the art, the number of individual cell culture aliquots required will depend on the size of the aliquots and the total volume of test sample to be tested within the method. A person of skill in the art would be able to determine an appropriate number of individual cell culture aliquots with a maximum aqueous volume of less than 12 ml for their desired purpose.

For example, when using 24-well plate, a maximum aqueous volume of 0.6 ml per well may be used.

For example, the FDA guidelines for RCLCC testing state that 1% or a maximum of 1×10⁸ end-of-production Cells (EOPCs) should be tested. Under standard testing conditions, a person of skill in the art may carry this out by co-cultivation of EOPCs with a virus permissive cell line (e.g. C8166 cells), cell passaging and analysis of the harvest supernatants (e.g. by F-PERT). Using conventional flask-based assays, ten T225 flasks may be individually seeded with 1.00 E+07 C8166 cells and 1.00 E+07 EOPCs in a total volume of 40 ml. Thus, the initial seeding density of the RCLCC assay would be 5.00 E+05 cells per ml.

In order to comply with the FDA guidelines using the methods described herein, a person of skill in the art would realise that, based on the seeding density used in the flask-based assay above, 28× 24-well plates may be used to perform the RCLCC assay at 24-well plate scale, using a maximum aqueous volume of 0.6 ml per well. A person of skill in the art would therefore be able to select an appropriate number of aliquots with an appropriate culture volume (and at an appropriate seeding density) in order to perform their desired method using the parameters set out herein.

For the avoidance of doubt, a similar methodology may be used to determine the number of aliquots and corresponding volumes needed for other assays such as RCL assays (or any other replication competent viral assays).

In one example, the total volume of the plurality of individual cell culture aliquots of step a) may be at least about 115 ml. The total volume may be within one cell culture vessel (e.g. when only one cell culture plate is used, where the total volume is the sum of all of the aliquots present in the plate) or may be spread over more than one cell culture vessel (e.g. when more than one cell culture plate is used, where the total volume is the sum of all of the aliquots present in all of the plates).

For example, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc).

Therefore, for example, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 10 ml or less. In this example, the aqueous volume of the individual cell culture aliquots may each be 10 ml or less and the total volume of all the aliquots may be at least about 115 ml.

Alternatively, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 5 ml or less. In this example, the aqueous volume of the individual cell culture aliquots may each be 5 ml or less and the total volume of all the aliquots may be at least about 115 ml.

For example, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 3 ml or less. In this example, the aqueous volume of the individual cell culture aliquots may each be 3 ml or less and the total volume of all the aliquots may be at least about 115 ml.

In one example, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 2 ml or less. In this example, the aqueous volume of the individual cell culture aliquots may each be 2 ml or less and the total volume of all the aliquots may be at least about 115 ml.

In a further example, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 1 ml or less. In this example, the aqueous volume of the individual cell culture aliquots may each be 1 ml or less and the total volume of all the aliquots may be at least about 115 ml.

In one example, in the context of testing a sample that comprises end of production cells, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 0.6 ml or less. In this example, the aqueous volume of the individual cell culture aliquots may each be 0.6 ml or less and the total volume of all the aliquots may be at least about 115 ml.

For example, in the context of testing a sample that comprises viral particles (e.g. harvest supernatant), the total volume of the plurality of individual cell culture aliquots of step a) may be at least 30 ml (for example at least 40 ml, at least 50 ml, at least 60 ml, at least 70 ml, at least 80 ml, at least 90 ml etc). For example, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc).

Therefore, for example, in the context of testing a sample that comprises viral particles, the total volume of the plurality of individual cell culture aliquots of step a) may be at least 30 ml (for example at least 40 ml, at least 50 ml, at least 60 ml, at least 70 ml, at least 80 ml, at least 90 ml etc), wherein the aqueous volume of the individual cell culture aliquots may each be 10 ml or less. In this example, the aqueous volume of the individual cell culture aliquots may each be 10 ml or less and the total volume of all the aliquots may be at least about 50 ml.

Alternatively, in the context of testing a sample that comprises viral particles, the total volume of the plurality of individual cell culture aliquots of step a) may be at least 30 ml (for example at least 40 ml, at least 50 ml, at least 60 ml, at least 70 ml, at least 80 ml, at least 90 ml etc), wherein the aqueous volume of the individual cell culture aliquots may each be 5 ml or less. In this example, the aqueous volume of the individual cell culture aliquots may each be 5 ml or less and the total volume of all the aliquots may be at least about 50 ml.

For example, in the context of testing a sample that comprises viral particles, the total volume of the plurality of individual cell culture aliquots of step a) may be at least 30 ml (for example at least 40 ml, at least 50 ml, at least 60 ml, at least 70 ml, at least 80 ml, at least 90 ml etc), wherein the aqueous volume of the individual cell culture aliquots may each be 3 ml or less. In this example, the aqueous volume of the individual cell culture aliquots may each be 3 ml or less and the total volume of all the aliquots may be at least about 50 ml.

In one example, in the context of testing a sample that comprises viral particles, the total volume of the plurality of individual cell culture aliquots of step a) may be at least 30 ml (for example at least 40 ml, at least 50 ml, at least 60 ml, at least 70 ml, at least 80 ml, at least 90 ml etc), wherein the aqueous volume of the individual cell culture aliquots may each be 2 ml or less. In this example, the aqueous volume of the individual cell culture aliquots may each be 2 ml or less and the total volume of all the aliquots may be at least about 50 ml.

In a further example, in the context of testing a sample that comprises viral particles, the total volume of the plurality of individual cell culture aliquots of step a) may be at least 30 ml (for example at least 40 ml, at least 50 ml, at least 60 ml, at least 70 ml, at least 80 ml, at least 90 ml etc), wherein the aqueous volume of the individual cell culture aliquots may each be 1 ml or less. In this example, the aqueous volume of the individual cell culture aliquots may each be 1 ml or less and the total volume of all the aliquots may be at least about 50 ml.

In one example, in the context of testing a sample that comprises viral particles, the total volume of the plurality of individual cell culture aliquots of step a) may be at least 30 ml (for example at least 40 ml, at least 50 ml, at least 60 ml, at least 70 ml, at least 80 ml, at least 90 ml etc), wherein the aqueous volume of the individual cell culture aliquots may each be 0.6 ml or less. In this example, the aqueous volume of the individual cell culture aliquots may each be 0.6 ml or less and the total volume of all the aliquots may be at least about 50 ml.

Alternatively, for example, in the context of testing a sample that comprises viral particles, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 10 ml or less. In this example, the aqueous volume of the individual cell culture aliquots may each be 10 ml or less and the total volume of all the aliquots may be at least about 115 ml.

For example, in the context of testing a sample that comprises viral particles, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 5 ml or less. In this example, the aqueous volume of the individual cell culture aliquots may each be 5 ml or less and the total volume of all the aliquots may be at least about 115 ml.

Alternatively, in the context of testing a sample that comprises viral particles, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 3 ml or less. In this example, the aqueous volume of the individual cell culture aliquots may each be 3 ml or less and the total volume of all the aliquots may be at least about 115 ml.

In one example, in the context of testing a sample that comprises viral particles, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 2 ml or less. In this example, the aqueous volume of the individual cell culture aliquots may each be 2 ml or less and the total volume of all the aliquots may be at least about 115 ml.

In a further example, in the context of testing a sample that comprises viral particles, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 1 ml or less. In this example, the aqueous volume of the individual cell culture aliquots may each be 1 ml or less and the total volume of all the aliquots may be at least about 115 ml.

In one example, in the context of testing a sample that comprises viral particles, the total volume of the plurality of individual cell culture aliquots of step a) may be at least 100 ml (e.g. 100 ml or more, 110 ml or more, 115 ml or more, 120 ml or more, 130 ml or more, 140 ml or more, 150 ml or more, 200 ml or more, 250 ml or more, 300 ml or more, 350 ml or more, 400 ml or more, 450 ml or more etc), wherein the aqueous volume of the individual cell culture aliquots may each be 0.6 ml or less. In this example, the aqueous volume of the individual cell culture aliquots may each be 0.6 ml or less and the total volume of all the aliquots may be at least about 115 ml.

As will be clear to a person of skill in the art, the total volume of the plurality of individual cell culture aliquots required for step a) will be depend on the total number of cells needed in step a) and the initial seeding density that is used in the plurality of individual cell culture aliquots of step a). A person of skill in the art will be able to adjust these parameters as appropriate for their desired purpose.

The inventors have identified that, when using a plurality of individual cell culture aliquots each with a maximum aqueous volume of less than 12 ml (for example, a maximum aqueous volume of 11 ml, 10 ml, 5 ml or 3 ml or less as appropriate), an initial seeding density of at least 1 × 10⁵ total cells/ml can be used. As used herein, “seeding density” refers to the total number of cells per unit volume that is added to the cell culture vessel in order to seed the vessel with cells. In the context of the present invention, “initial seeding density” refers to the number of cells per unit volume provided in step a). Suitable seeding densities according to the methods of the present invention are provided elsewhere herein.

Typically, at the start of culture in the methods described herein, the density of cells (initial seeding density) that is present in the aliquots can be in the range of from about 1 × 10⁵ total cells/ml to about 1 × 10⁷ total cells/ml. In this context “total cells/ml” is used to refer to all of the cells in the aliquot (irrespective of whether they are cells from the test sample (e.g. end of production cells) or virus-permissive cells). This seeding density is roughly equivalent to that which is used in conventional flask-based culturing methods.

Accordingly, in one example, the initial seeding density of the plurality of individual cell culture aliquots in step a) is at least 1 × 10⁵ total cells/ml, at least 2 × 10⁵ total cells/ml, at least 3 × 10⁵ total cells/ml, at least 4 × 10⁵ total cells/ml, at least 5 × 10⁵ total cells/ml, at least 6 × 10⁵ total cells/ml, at least 7 × 10⁵ total cells/ml, at least 8 × 10⁵ total cells/ml, at least 9 × 10⁵ total cells/ml, at least 1 × 10⁶ total cells/ml, at least 2 × 10⁶ total cells/ml, at least 3 × 10⁶ total cells/ml, at least 4 × 10⁶ totalcells/ml, at least 5 × 10⁶ total cells/ml, at least 6 × 10⁶ total cells/ml, at least 7 × 10⁶ total cells/ml, at least 8 × 10⁶ total cells/ml, at least 9 × 10⁶ total cells/ml, or at least 1 × 10⁷ total cells/ml.

Accordingly, in another example, the initial seeding density of the plurality of individual cell culture aliquots in step a) is in the range of from about 1 × 10⁵ total cells/ml to about 1 × 10⁷ total cells/ml.

For example, the initial seeding density of the plurality of individual cell culture aliquots in step a) is in the range of from about 5 × 10⁵ total cells/ml to about 1 × 10⁷ total cells/ml.

In another example, the initial seeding density of the plurality of individual cell culture aliquots in step a) is in the range of from about 1 × 10⁶ total cells/ml to about 1 × 10⁷ total cells/ml.

The plurality of individual cell culture aliquots each comprise a portion of the test sample (i.e. a percentage of the total sample being tested) and virus-permissive cells. Virus-permissive cells are cells that can support the growth of a virus and permit viral replication. A permissive cell or host is one that allows a virus to circumvent its defences and replicate. The type of virus-permissive cell for use in the methods described herein is typically chosen based on the virus of interest (i.e. the virus of interest and the virus-permissive cell are chosen to be compatible). Non-limiting examples of appropriate virus-permissive cells include: immortalised T cell lines such as C8166 cells (permissive to HIV for example) and non T cell lines such as HEK293 cells (permissive to MLV and EIAV for example). A person of skill in the art can readily identify appropriate virus-permissive cells for the virus of interest.

In one example, the virus-permissive cells are immortalised T cell lines. Appropriate T cell lines include Jurkat, CEM-SS, PM1, Molt4, Molt4.8, SupT1, MT4 or C8166 cells.

In an alternative example, the virus-permissive cells are non-T cell lines. Appropriate non T cell lines include HEK293 or 92BR cells.

In one example, the virus-permissive cells are non-adherent. As used herein “non-adherent cells” are cells that do not attach to a surface. For the avoidance of doubt, non-adherent cells may form cellular aggregates within a cell culture aliquot. Many cell types grow in solution and not attached to a surface. Non-adherent cells can be sub-cultured by simply taking a small volume of the parent culture and diluting it in fresh growth medium. Cell density in these cultures is normally measured in cells/ml. The cells will often have a preferred range of densities for optimal growth and subculture (referred to as “passaging” herein) will normally try to keep the cells in this range. Use of non-adherent cells in the methods described herein is particularly advantageous, for example when the methods are automated.

A non-limiting example of a non-adherent virus-permissive immortalised T cell line is a C8166 cell. C8166 cells are typically used in the methods described herein when a virus (e.g. HIV or equivalent) for which C8166 is permissive is being detected.

In an alternative example, the virus-permissive cells are adherent. Adherent cells grow attached to a surface such as the bottom of the cell culture vessel. These cell types have to be detached from the surface before they can be sub-cultured. For subculture cells may be detached by one of several methods including trypsin treatment to break down the proteins responsible for surface adherence, chelating calcium ions with EDTA which disrupts some protein adherence mechanisms, or mechanical methods like repeated washing or use of a cell scraper. The detached cells are then resuspended in fresh growth medium and allowed to settle back onto their growth surface.

A non-limiting example of adherent virus-permissive cells is HEK293 cells (which are a non T cell line). HEK293 cells are typically used in the methods described herein when a virus (e.g. EIAV or equivalent) for which HEK293 is permissive is being detected). In some examples, HEK293 cells may also be considered to be non-adherent, as they can be adapted to be in suspension.

The step of providing a plurality of individual cell culture aliquots may include generating the plurality of individual cell culture aliquots from a source sample. In other words, the method may include the step of mixing a source test sample with virus permissive cells and dividing it into aliquots so as to generate the individual cell culture aliquots. Accordingly, a single mixture of test sample and virus permissive cells may be provided initially and then aliquoted to provide a plurality of individual cell culture aliquots. Alternatively, the method may include the step of mixing a portion of a source test sample with a portion of virus permissive cells to generate each individual cell culture aliquot separately.

### Culturing the Aliquots

The methods described herein include culturing the cell culture aliquots. The term “culturing” as used herein refers to keeping cells in an artificial (e.g. in vitro or ex vivo) environment. Typically, cells are cultured under conditions favouring their proliferation, differentiation, and/or continued viability. The cells are typically cultured in a cell culture medium.

The terms “cell culture medium” and “culture medium” (plural “media” in each case) refer to a nutritive solution for cultivating live cells. Various cell culture media will be known to those skilled in the art, who will also appreciate that the type of cells to be cultured may dictate the type of culture medium to be used.

For example, the culture medium may be selected from the group consisting of Dulbecco’s Modified Eagle’s Medium (DMEM), Ham’s F-12 (F-12), Minimal Essential Medium (MEM), Basal Medium Eagle (BME), RPMI-1640, Ham’s F-10, αMinimal Essential Medium (αMEM), Glasgow’s Minimal Essential Medium (G-MEM), and Iscove’s Modified Dulbecco’s Medium (IMDM), or any combination thereof. Other media that are commercially available (e.g., from Thermo Fisher Scientific, Waltham, MA) or that are otherwise known in the art can be equivalently used in the context of this disclosure. Again, only by way of example, the media may be selected from the group consisting of 293 SFM, CD-CHO medium, VP SFM, BGJb medium, Brinster’s BMOC-3 medium, cell culture freezing medium, CMRL media, EHAA medium, eRDF medium, Fischer’s medium, Gamborg’s B-5 medium, GLUTAMAX™ supplemented media, Grace’s insect cell media, HEPES buffered media, Richter’s modified MEM, IPL-41 insect cell medium, Leibovitz’s L-15 media, McCoy’s 5A media, MCDB 131 medium, Media 199, Modified Eagle’s Medium (MEM), Medium NCTC-109, Schneider’s Drosophila medium, TC-100 insect medium, Waymouth’s MB 752/1 media, William’s Media E, protein free hybridoma medium II (PFHM II), AIM V media, Keratinocyte SFM, defined Keratinocyte SFM, STEMPRO® SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX® bone marrow karyotyping medium, and KNOCKOUT™ D-MEM, or any combination thereof.

The methods comprise culturing the aliquots for an appropriate duration of time. Typically, when cells are cultured for a duration of at least two days, it is beneficial to passage the cells into fresh medium. As used herein, a “passage” refers to the step of harvesting grown cells from one “parent” cell culture aliquot (also referred to as a “parent aliquot” herein) and reseeding them to generate a new “daughter” cell culture aliquot (also referred to as a “daughter aliquot” herein). In other words, it refers to the sub-culture of cell cultures. In this context, the daughter aliquot is the new aliquot into which the cells are being sub-cultured and the parent aliquot is the previous aliquot that is being passaged or sub-cultured. Accordingly, passaging refers to the transfer of a proportion of cell suspension and/or supernatant from an aliquot to another.

When adherent cells are passaged, the cells are typically washed in PBS while still adherent, detached from the aliquot and then resuspended in media. A proportion of the resuspended cells are transferred to a new aliquot. When non-adherent cells are passaged, the cells are in suspension so a proportion of an aliquot can be directly transferred to a new aliquot.

The passage number of a cell culture refers to the number of times it has been harvested and reseeded. During passage, a volume of the parent cell culture aliquot is harvested and re-seeded in the new daughter aliquot (typically into fresh cell culture medium). The volume of the parent aliquot that is re-seeded in the daughter aliquot may be characterised by the dilution factor used when harvesting the cells from the parent aliquot and re-seeding the cells to generate the daughter aliquot. Alternatively, it may be characterised as a percentage of the cells from the parent cell culture aliquot, or as the initial cell seeding density of the daughter aliquot.

Culturing the aliquots in step b)

In step b) of the methods described herein the aliquots are cultured for at least nine days. For example, the aliquots may be cultured within step b) of the method for at least 9 days, at least 10 days, at least 11 days, or at least 12 days etc.

In the context of the method described herein, step b) of the method may therefore include at least one passage, wherein the cells from a parent aliquot are harvested and re-seeded into a new daughter aliquot. Standard methods for passaging cells are well known in the art.

Typically, “direct passages” are used when passaging the aliquots in step b). As used herein, a “direct passage” refers to a passage wherein the cells in one daughter aliquot are derived from one parent aliquot. The terms “direct passage” and “serial passage” are used interchangeably herein. In direct passages, the total number of aliquots between each generation (parent to daughter) therefore remains constant, although the cell number in parent vs daughter aliquots will be different (due to the dilution factor that occurs during passage, wherein only a proportion of the cells in the parent cell culture aliquot are transferred to the daughter aliquot).

In other words, “direct passaging” refers to passages performed without the pooling of aliquots i.e. volume X is transferred from aliquot 1 to aliquot 2. The table below demonstrates some examples of how splitting ratios and dilution factors may used in the context of direct passaging.

TABLE 1

| Starting Plate Format | Starting Aliquot Volume (µl) | Volume Transferred (µl) | Splitting Ratio (DF) | Final Plate Format | Final Aliquot Volume (µl) | Dilution Factor |
|---|---|---|---|---|---|---|
| 24-Well | 1000 | 500 | 2 | 24-Well | 1000 | 2 |
| | 1000 | 250 | 4 | 24-Well | 1000 | 4 |
| | 1000 | 100 | 10 | 24-Well | 1000 | 10 |
| | 1000 | 500 | 2 | 12-Well | 2000 | 4 |
| | 1000 | 250 | 4 | 12-Well | 2000 | 8 |
| | 1000 | 100 | 10 | 12-Well | 2000 | 20 |
| 12-Well | 2000 | 1000 | 2 | 12-Well | 2000 | 2 |
| | 2000 | 500 | 4 | 12-Well | 2000 | 4 |
| | 2000 | 250 | 8 | 12-Well | 2000 | 8 |

Examples of direct passaging of aliquots. As used herein a “splitting ratio” is the proportion of cell suspension and/or supernatant transferred from one aliquot to another. By contrast, a “dilution factor” takes into account the final aliquot volume. If the final aliquot volume is larger than the starting aliquot, this should also be taken into account.

Accordingly, step b) of the methods described herein may comprise culturing the aliquots for at least nine days, wherein the aliquots are directly passaged during the at least nine days. In one example, step b) of the methods described herein may comprise culturing the aliquots for at least nine days, wherein the aliquots are directly passaged at least twice during the at least nine days.

Typically (but not always), during direct passaging of aliquots, the total volume of a daughter aliquot is equivalent to (or the same as) the total volume of the parent aliquot from which it is derived. In other words, if the parent aliquot has a total volume of 3 ml, then the daughter aliquot typically also has a total volume of 3 ml. In such cases, the total volume across all of the parent aliquots is the same as the total volume across all of the daughter aliquots.

Typically, during direct passaging of aliquots, not all of the cells from the parent aliquot are transferred into the daughter aliquot. For example, a splitting ratio of at least 2 to 20 may be used, for example, a splitting ratio of at least 4, at least 5, at least 6, at least 8, at least 10, at least 12, at least 14, at least 16, at least 18, up to 20 may be used. A “splitting ratio” refers to the proportion of cell suspension and/or supernatant transferred from the parent aliquot to the daughter aliquot. A splitting ratio of 2 may therefore be considered equivalent to 50% of the cell suspension and/or supernatant in the parent cell culture aliquot being re-seeded in the new “daughter” aliquot. Similarly, a splitting ratio of 4 may therefore be considered equivalent to 25% of the cell suspension and/or supernatant in the parent cell culture aliquot being re-seeded in the new “daughter” aliquot. Furthermore, a splitting ratio of 20 may therefore be considered equivalent to 5% of the cell suspension and/or supernatant in the parent cell culture aliquot being re-seeded in the new “daughter” aliquot. In the examples provided below, a splitting ratio of 4 was used during step b) of the method. However, it will be appreciated that different splitting ratios may be suitable for different cell types or different culture conditions.

Accordingly, step b) of the methods described herein may comprise culturing the aliquots for at least nine days, wherein the aliquots are directly passaged during the at least nine days, and a splitting ratio of at least 2 (e.g. at least 4) is used for each passage, optionally wherein the total volume across all of the parent aliquots is the same as the total volume across all of the daughter aliquots for each passage. The splitting ratio may be in the range of from about 2 to about 20, for example.

Appropriate initial seeding densities are discussed elsewhere herein (e.g. in the contest of step a)) and apply equally here. These initial seeding densities may also be used as a guide for the number of cells needed for re-seeding of each daughter aliquot during passaging of step b) (and thus the appropriate splitting ratio that can be used). For example, initial seeding densities for the daughter aliquots of each passage may be in the range of from about 1 × 10⁵ total cells/ml to about 1 × 10⁷ total cells/ml. For example, initial seeding densities for the daughter aliquots of each passage may be in the range of from about 5 × 10⁵ total cells/ml to about 1 × 10⁷ total cells/ml etc.

### Culturing and Passaging Using Dilution Factors in Step C

The methods described herein comprise step c), wherein the aliquots are cultured for at least a further six days after step b). During step c), the aliquots are passaged using a dilution factor of at least 2 at each passage. In other words, each aliquot in step c) is passaged by diluting the parent aliquot by at least 2 to generate the daughter aliquot. As used herein, the term “dilution factor” refers to the ratio of the volume of the initial solution (the volume transferred from the parent aliquot) to the volume of the final solution (daughter aliquot), that is, the ratio of V₁ to V₂. or V₁: V₂. A dilution factor, DF, can be calculated: DF = V₂ ÷ V₁. For example, when 500 µl of the parent aliquot is used to generate a daughter aliquot with a total volume of 1 ml, this represents a dilution factor (DF) of 2. As a further example, when 250 µl of the parent aliquot is used to generate a daughter aliquot with a total volume of 1 ml, this represents a dilution factor (DF) of 4 etc. Other appropriate dilution factors can be identified by a person of skill in the art.

A dilution factor of 2 is also referred to in the art as a dilution factor of 1:2 or a dilution factor of 1 to 2. This refers to combining one unit volume from the parent aliquot with one new unit volume (e.g. of new culture medium) to generate a new daughter aliquot with a total of 2 unit volumes. Similarly, a dilution factor of 4 is also referred to in the art as a dilution factor of 1:4 or a dilution factor of 1 to 4. This refers to combining one unit volume from the parent aliquot with three new unit volumes (e.g. of new culture medium) to generate a new daughter aliquot with a total of 4 unit volumes. Similarly, a dilution factor of 8 is also referred to in the art as a dilution factor of 1:8 or a dilution factor of 1 to 8. This refers to combining one unit volume from the parent aliquot with seven new unit volumes (e.g. of new culture medium) to generate a new daughter aliquot with a total of 8 unit volumes.

In some examples, the aliquots are passaged using a dilution factor of at least 2 at each passage. In some examples, the aliquots are passaged using a dilution factor of at least 4 at each passage. In some examples, the aliquots are passaged using a dilution factor of at least 6 at each passage.

In some examples, the aliquots are passaged using a dilution factor of at least 8 at each passage that takes place in step c).

In some examples, the aliquots are passaged using a dilution factor in the range of from about 2 to about 20 at each passage that takes place in step c).

The number of passages that will take place during step c) will vary, depending on the overall duration of step c). The appropriate number of passages can be readily determined by a person of skill in the art, using their common general knowledge. For example, step c) may include two passages, three passages or more (for example, if the duration of step c) is more than six days).

The methods provided herein are advantageous as they use a dilution factor of at least 2 (e.g. in the range of from about 2 to about 20), together with low individual aliquot volumes (of less than 12 ml). The methods described herein may additionally include one or more of the following features in step c):
(i) a reduction in total volume across the aliquots (when comparing total volume at the start of step c) to total volume at the end of step c))
(ii) pooling of aliquots to reduce the overall aliquot number (when comparing total aliquot number at the start of step c) to total aliquot number at the end of step c))
(iii) using a split ratio of at least 4 during passage in step c)
(iv) using a combination of (i) and (ii)
(v) using a combination of (i) and (iii)
(vi) using a combination of (ii) and (iii)
(vii) using a combination of all of (i), (ii) and (iii).

Each of these aspects is discussed below individually. All features discussed below individually are also applicable to the combinations described herein. These aspects are particularly useful when used with small aliquot volumes such as volumes of 3 ml or less because they facilitate automation.

### (I) Volume of Aliquots in Step C

During passaging in step c), the total volume of a daughter aliquot may be equivalent to (or the same as) the total volume of the parent aliquot from which it is derived. For example, if the parent aliquot has a total volume of 3 ml, then the daughter aliquot may also have a total volume of 3 ml.

Alternatively, during passaging in step c), the total volume of a daughter aliquot may be different (e.g. higher, but preferably lower) than the total volume of the parent aliquot from which it is derived. In other words, if the parent aliquot has a total volume of 3 ml, then the daughter aliquot may have a total volume that is different (e.g. higher, but preferably lower) than 3 ml.

In some examples, the total volume across all of the aliquots at the end of step c) is the same or smaller than the total volume across all of the aliquots at the start of step c). This may be achieved by reducing the volume of daughter aliquots (compared to parent aliquots) during passaging or by pooling parent aliquots during passaging to generate a daughter aliquot with two parents. A reduction in total volume across the aliquots is particularly advantageous in methods for detecting replication competent virus, which typically use large overall culture volumes (and thus can be laborious to perform). For example, the total volume across all of the aliquots may be reduced by at least 50% during step c). It may be reduced by at least 75%, at least 83%, at least 87.5%, at least 90% etc. In a particular example, the total volume across all of the aliquots may be reduced by at least 87.5% during step c).

In other words, step c) may comprise culturing the aliquots for at least a further six days, wherein the aliquots are passaged using a dilution factor of at least 2 at each passage (e.g. in the range of from about 2 to about 20), and wherein the total volume across all of the aliquots is reduced by at least 50% or at least 87.5% during step c) (e.g. over at least two passages, or at least three passages).

As stated above, the total volume across all of the aliquots at the end of step c) may be reduced compared to that at the start of step c) by reducing the volume of daughter aliquots (compared to parent aliquots) during passaging or by pooling parent aliquots during passaging.

### (II) Pooling Passages in Step C

Pooling passages may be particularly advantageous in step c) of the method described herein as they can be used to reduce the total aliquot number over the passages used in step c) (and may additionally be used to reduce the volume across the aliquots by the end of step c)) which can make liquid handling easier. As used herein, a “pooling passage” refers to harvesting parent aliquots and pooling parent aliquots such that one daughter aliquot is re-seeded with cells or supernatant from more than one parent aliquot. Pooling may include re-seeding one daughter aliquot with cells from two parents, three parents, or four parents etc.

For example, the cells from two parent aliquots may be harvested and combined (“pooled”) and a proportion of the combined cells may be used to re-seed one daughter aliquot. In another example, the cells from two parent aliquots may be harvested but not combined, and a proportion of each of the parent cells may be added to one daughter aliquot to effect pooling of the cells from those parents. Pooling therefore reduces the overall number aliquots after each passage.

TABLE 2

| Process | Starting Plate Format | Aliquot ID | Starting Aliquot Volume (µl) | Volume Transferred (µl) | Splitting Ratio (DF) | Final Plate Format | Final Aliquot Volume (µl) | Dilution Factor |
|---|---|---|---|---|---|---|---|---|
| 24 w Pool 24 w | 24-Well | A | 1000 | 250 | 4 | 24-Well | 1000 | 4 |
| | | B | 1000 | 250 | 4 | | | 4 |
| 24 w Pool 12 w | 24-Well | A | 1000 | 250 | 4 | 12-Well | 2000 | 8 |
| | | B | 1000 | 250 | 4 | | | 8 |
| 12 w Pool 12 w | 12-Well | A | 2000 | 500 | 4 | 12-Well | 2000 | 4 |
| | | B | 2000 | 500 | 4 | | | 4 |
| | 12-Well | A | 2000 | 250 | 8 | 12-Well | 2000 | 8 |
| | | B | 2000 | 250 | 8 | | | 8 |

Examples of pooling passaging of aliquots. i.e. volume X from aliquot A and volume Y from aliquot B are both transferred to aliquot C.

Accordingly, in some examples, pooling passages may be used in step c) to reduce the overall aliquot number by a pooling factor of at least 2. As used herein, the term “pooling factor” refers to the ratio of the total number of aliquots at the start of step c) (referred to as “A₁”) to the total number of aliquots at the end of step c) (referred to as “A₂”), that is, the ratio of A₁ to A₂. or A₁: A₂. A pooling factor, PF, can be calculated: PF = A₁ ÷ A₂. For example, when the total number of aliquots at the start of step c) is 96 and the total number of aliquots at the end of step c) is 24, then the pooling factor is 96 ÷ 24 = 4.

A pooling factor of 2 is also referred to herein as a pooling factor of 2:1 or a pooling factor of 2 to 1. This refers to pooling two parent aliquots into one daughter aliquot. Similarly, a pooling factor of 4 is also referred to herein as a pooling factor of 4:1 or a pooling factor of 4 to 1. This refers to pooling 4 parent aliquots into one daughter aliquot. Similarly, a pooling factor of 8 is also referred to herein as a pooling factor of 8:1 or a pooling factor of 8 to 1. This refers to pooling 8 parent aliquots into one daughter aliquot.

Pooling may be performed using a pairwise approach (wherein two parent aliquots are pooled into one daughter aliquot at the end of each passage). A pairwise approach to pooling is demonstrated in the examples section below. Other appropriate pooling methods may also be used, including for example, pooling more than two parent aliquots at a time. A non-limiting example of this may be pooling four parent aliquots into one daughter aliquot (e.g. pooling four aliquots from a 24 well plate into one aliquot on a 6 well plate). This approach allows operators to sequentially reduce the total number of aliquots (or assay plates) over the duration of the assay without compromising assay sensitivity i.e. an assay can be initiated with 8 plates, and sequentially reduced to a single plate over 3-4 weeks.

The aliquots may be pooled during step c) by a pooling factor in the range of from about 2 to about 8.

In some examples, the aliquots are pooled during step c) by a pooling factor of at least 2 (i.e. when comparing the number of aliquots at the start of step c) with the number of aliquots at the end of step c), the total number has reduced by a pooling factor of at least 2, for example over at least two passages, or over at least three passages). In some examples, the aliquots are pooled during step c) by a pooling factor of at least 4 (i.e. when comparing the number of aliquots at the start of step c) with the number of aliquots at the end of step c), the total number has reduced by a pooling factor of at least 4, for example over at least two passages, or over at least three passages). In some examples, the aliquots are pooled during step c) by a pooling factor of at least 6 (i.e. when comparing the number of aliquots at the start of step c) with the number of aliquots at the end of step c), the total number has reduced by a pooling factor of at least 6, for example over at least two passages, or over at least three passages).

In some examples, the aliquots are pooled during step c) by a pooling factor of at least 8 (i.e. when comparing the number of aliquots at the start of step c) with the number of aliquots at the end of step c), the total number has reduced by a pooling factor of at least 8, for example over at least two passages, or over at least three passages).

The aliquots may be pooled during each passage in step c) by a pooling factor in the range of from about 2 to about 8.

In some examples, the aliquots are pooled during each passage in step c) using a pooling factor of at least 2. In some examples, the aliquots are pooled during each passage in step c) using a pooling factor of at least 4. In some examples, the aliquots are pooled during each passage in step c) using a pooling factor of at least 6. In some examples, the aliquots are pooled during each passage in step c) using a pooling factor of at least 8. This may occur over at least two, or at least three passages.

In some examples therefore, step c) may comprise culturing the aliquots for at least a further six days, wherein the aliquots are passaged using a dilution factor of at least 2 at each passage, and wherein the aliquots are pooled during step c) to reduce the overall aliquot number at the end of step c) by a pooling factor of at least 4 or at least 8.

It may be particularly advantageous to reduce the overall aliquot number and the total volume across the aliquots during step c). Accordingly, in one example, step c) may comprise culturing the aliquots for at least a further six days, wherein the aliquots are passaged using a dilution factor of at least 2 at each passage, and wherein the aliquots are pooled to reduce the overall aliquot number by pooling factor of at least 4 or at least 8 during step c), wherein the total volume across all of the aliquots is also reduced by at least 50% or at least 87.5% during step c).

As described above in the context of step b), during passaging of aliquots, not all of the cells from the parent aliquot are transferred into the daughter aliquot. This also applies to passaging in step c). Accordingly, in step c) a splitting ratio of at least 2 to 20 may be used. Splitting ratios in the context of direct passaging is discussed in detail elsewhere herein. In the context of pooling passages, a “splitting ratio” is calculated for each parent individually (as the proportion of cell suspension and/or supernatant from that parent aliquot that is transferred to the daughter aliquot).

Suprisingly, the inventors have found that splitting ratios used in step c) may be higher than those used in step b) (especially in the context of pooling passages) without adversely affecting sensitivity of the method. Accordingly, a splitting ratio of at least 4 is preferred in step c) (especially in the context of pooling passages).

### Passaging With Splitting Ratios in Step C

During step c) of the methods described herein, the aliquots are passaged using a dilution factor of 2 at each passage. During passaging, any appropriate splitting ratio may be used. As discussed elsewhere herein, appropriate splitting ratios include 4 to 20.

Suprisingly, the inventors have found that splitting ratios used in step c) may be higher than those used in step b) without adversely affecting sensitivity of the method, even when small aliquot volumes of less than 12 ml (e.g. 3 ml or less) are used. In other words, a significant proportion of the parent aliquot can be discarded at each passage without adversely affecting the overall sensitivity of the assay. Accordingly, in one example, step c) may comprise culturing the aliquots for at least a further six days, wherein the aliquots are passaged using a dilution factor of at least 2 and a splitting ratio of at least 4 at each passage. Optionally, the total volume across all of the aliquots may simultaneously be reduced by at least 50% or at least 87.5% during step c).

When such splitting ratios are used, the total volume of a daughter aliquot may be equivalent to (or the same as) the total volume of the parent aliquot from which it is derived. In other words, if the parent aliquot has a total volume of 3 ml, then the daughter aliquot may also have a total volume of 3 ml. Alternatively, during passaging in step c), the total volume of a daughter aliquot may be different (e.g. higher, but preferably lower) than the total volume of the parent aliquot from which it is derived. In other words, if the parent aliquot has a total volume of 3 ml, then the daughter aliquot may have a total volume that is different (e.g. higher, but preferably lower) than 3 ml.

In some examples, the total volume across all of the aliquots at the end of step c) is the same or smaller than the total volume across all of the aliquots at the start of step c) when splitting ratios of at least 4 are used. This may be achieved by reducing the volume of daughter aliquots (compared to parent aliquots) during passaging or by pooling parent aliquots during passaging. A reduction in total volume across the aliquots is particularly advantageous in methods for detecting replication competent virus, which typically use large overall culture volumes (and thus can be laborious to perform). For example, the total volume across all of the aliquots may be reduced by at least 50% during step c). It may be reduced by at least 75%, at least 83%, at least 87.5%, at least 90% etc. In a particular example, the total volume across all of the aliquots may be reduced by at least 87.5% during step c).

During passages the re-seeding cell number should also be taken into account. Appropriate initial seeding densities are discussed elsewhere herein (e.g. in the contest of step a)) and apply equally here. These initial seeding densities may also be used as a guide for the number of cells needed for re-seeding of each daughter aliquot during passaging of step b). For example, initial seeding densities for the daughter aliquots of each passage may be in the rage of from about 1 × 10⁵ total cells/ml to about 1 × 10⁷ total cells/ml. For example, initial seeding densities for the daughter aliquots of each passage may be in the range of from about 5 × 10⁵ total cells/ml to about 1 × 10⁷ total cells/ml etc.

### Duration of Culture in Step C

Step c) of the methods described herein comprises culturing the aliquots for at least a further six days. For the avoidance of doubt, the aliquots may be cultured for a longer duration, for example for at least a further seven, eight, nine, ten, eleven or more days before testing for replication competent virus. The methods described herein may therefore take at least 3 weeks, e.g. 3 to 4 weeks or 4 to 5 weeks to complete.

In examples where the test sample comprises cells (e.g. end of production cells) it may be beneficial to include an additional filtration step (e.g. using a 0.45 µm filter) at some point before testing for replication competent virus (i.e. before step d) of the methods described herein).

Advantageously, the methods described herein may be automated. As used herein “automated” refers a technique, method, or system of operating or controlling the method by highly automatic means, including by electronic devices. Automation of the method may reduce the workload of the operator or increase the throughput of the method. A non-limiting example of an automatic means that may be used in the automated method is a liquid handler. Appropriate liquid handlers are known in the art. Advantageously, automated methods can increase reliability of the methods described herein.

For the avoidance of doubt, it may be that step b) and c) only are automated. Optionally, step a) and/or step d) may also be automated. Steps b) and c) may be automated separately to step d). For example, some operator interaction and/or input may be required to move from step c) to step d).

Any (or all) of the steps provided herein may also be performed manually. For example, step a) may be performed manually, with steps b) and c) (and optionally d)) being automated.

The method may be performed in parallel with a number of controls. Controls may include negative controls and/or positive controls.

An example of a negative control may be performing the method with aliquots that comprise virus-permissive cells (and all of the appropriate reagents etc), but no test sample. In this context, the method may be referred to as a “negative control method”. Suitably, the negative control method would be performed in parallel with the method for detecting a replication competent virus in a test sample, using the same reagents, culture conditions, virus permissive cells, pooling strategy, detection means etc.

An example of a positive control may be performing the method with aliquots that comprise virus-permissive cells and a replication competent virus (as a replacement of the test sample). In this context, the replication competent virus (“positive control”) may be referred to as being comprised within a “positive control sample” and the method may be referred to as a “positive control method”. Suitably, the positive control method would be performed in parallel with the method for detecting a replication competent virus in a test sample, using the same reagents, culture conditions, virus permissive cells, pooling strategy, detection means etc.

Typically (but not always), the positive control virus will be derived from the same virus from which the vector system (being tested in the RCR/RCL assay) is based. For example, but not by way of limitation, the positive control virus for SIV vectors will be derived from SIV, the positive control virus for HIV virus will be derived from HIV, etc (although more recently positive controls from MLV are increasingly being used as a more generic positive control). Ideally, the genome of the positive control virus will be functionally attenuated in all genes that are superfluous to replication competence within the chosen amplification/indicator cell line used in the RCR/RCL assay. For positive control viruses derived from lentiviruses, the attenuated genes are typically the accessory genes known for host/immune regulation/escape. This is because these gene/functions are typically absent from the retroviral/lentiviral vector system being employed, and so a putative RCR/RCL theoretically generated from the vector production process is extremely unlikely to acquire these functions. Auxiliary lentiviral genes such as tat or rev are typically maintained within the positive control virus genome, as these are typically essential for replication. Alternatively, MLV is used as the positive control virus it is a simple retrovirus lacking many of the specialised accessory genes present within lentivirus genomes, and most closely models a putative RCR/RCL likely to emerge from highly engineered, contemporary retroviral/lentiviral vector systems. Therefore, ideally a positive control virus lacking all accessory genes will be chosen but this may empirically depend on the efficiency of replication within the amplification/indicator cell line. Consequently, in order to develop robust RCR/RCL assays, sometimes the positive control virus will still express one or more functional accessory genes within the amplification/indicator cell line.

In one example, the positive control sample may comprise an attenuated replication competent lentivirus that has at least one accessory gene functionally deleted within its nucleotide sequence, wherein the at least one accessory gene is selected from: vif, vpr, vpx, vpu and nef. For example, the attenuated replication competent lentivirus may have at least three of vif, vpr, vpx, vpu and nef functionally deleted.

In a particular example, the positive control attenuated replication competent virus may comprise or consist of a nucleic acid sequence according to SEQ ID NO: 1, or be a variant thereof. This positive control is particularly useful in as a positive control for methods that are used to detect replication competent lentivirus in a test sample (especially when detecting replication competent HIV, SIV, SHIV or variants thereof), because it is particularly effective at remaining infectious over several passages (due to its vif+ status).

The variant may be a codon optimised variant of SEQ ID NO:1. As used herein “codon-optimised” (or “c.o.”) refers to polynucleotide sequences encoding the genes of interest that are modified relative to the native polynucleotide sequence whilst not altering the encoding amino acid sequence. This term is widely known in the art. Codon optimisation of a polynucleotide sequence can lead to several effects that increase overall translational efficiency/expression levels of the encoded proteins in a cell.

The variant may also be a functional variant of SEQ ID NO:1. Functional variants will typically contain only conservative substitutions of one or more amino acids, or a substitution, deletion or insertion of non-critical amino acids in non-critical regions of the protein(s) encoded by SEQ ID NO:1. Methods for identifying functional and non-functional variants (e.g. functional and non-functional allelic variants) are well known to a person of ordinary skill in the art.

A functional variant may comprise an nucleic acid sequence having at least about 60%, 65%, 70%, 75%, 80%, 85%, 90%, 91%, 92%, 93%, 94%, 95%, 96%, 97%, 98%, 99% or 100% identity to the nucleic acid sequence of SEQ ID NO:1. Suitably, percent identity can be calculated as the percentage of identity to the entire length of the reference sequence (e.g. SEQ ID NO: 1), or portions or fragments thereof.

A “non-essential” (or “non-critical”) amino acid residue is a residue that can be altered from the amino acid sequence encoded by SEQ ID NO:1 without abolishing or, more preferably, without substantially altering a biological activity, whereas an “essential” (or “critical”) amino acid residue results in such a change. For example, amino acid residues that are conserved are predicted to be particularly non-amenable to alteration, except that amino acid residues within the hydrophobic core of domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential (or non-critical) amino acid residue in a protein is preferably replaced with another amino acid residue from the same side chain family. Alternatively, in another embodiment, mutations can be introduced randomly, and the resultant mutants can be screened for biological activity to identify mutants that retain activity.

A conservative amino acid substitution variant of SEQ ID NO:1 may have at least one (e.g. two or fewer, three or fewer, four or fewer, five or fewer, six or fewer, seven or fewer, eight or fewer, nine or fewer, ten or fewer etc) conservative amino acid substitutions compared to the corresponding amino acid sequence encoded by SEQ ID NO:1.

The positive control discussed above may be useful in several different contexts besides the methods described herein. For example, it may be used as a positive control in conventional flask-based cell culture methods that are currently being used to detect replication competent virus. The positive control discussed above may therefore be useful in any RCL or RCLCC method. In this context, it is particularly useful in as a positive control for methods that are used to detect replication competent lentivirus in a test sample, for example when detecting replication competent HIV, SIV, SHIV or variants thereof.

The positive control described herein may be part of a kit. Suitably, a kit may further comprise one or more additional reagents, such as a buffer and the like. A buffer can be a stabilization buffer, a diluting buffer, or the like.

In addition to the above-mentioned components, a kit can further include instructions for using the components of the kit to practice the methods described herein. The instructions for practicing the methods are generally recorded on a suitable recording medium. For example, the instructions may be printed on a substrate, such as paper or plastic, etc. The instructions may be present in the kits as a package insert, in the labelling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this embodiment is a kit that includes a web address where the instructions can be viewed and/or from which the instructions can be downloaded. As with the instructions, this means for obtaining the instructions can be recorded on a suitable substrate.

The methods described herein include the step of testing for the presence of replication competent virus (step d) of the method). Any appropriate methods for detecting the presence of replication competent virus can be used. Typically, the step of testing for the presence of replication competent virus is performed once the passaging steps have been completed (e.g. after at least three weeks, at least four weeks or at least 5 weeks of culture), however test samples can also be collected from residual samples at each passage. However, it may additionally (or alternatively) also be performed before all of the passaging steps have been completed (e.g. at intermediate steps of the method). For example, it may be performed after 15 days, after 18 days, after 21 days, after 24 days, after 27 day, after 30 days, after 33 days or longer. It may therefore be performed more than once (e.g. at least two, at least three, at least four, at least five, at least six etc times during the method). In this context, it may be that supernatant is harvested at the desired time point(s) and stored until the method is completed such that all supernatants (representing different time points in the method) may be tested for replication competent virus at the same time (or in parallel). Appropriate means for obtaining and/or storing the supernatant are well known in the art.

For example, the presence of replication competent virus may be tested using PCR. In one example, RNA or DNA levels of a target gene e.g. psi-gag are measured using PCR (e.g. qPCR) as a means for detecting replication competent virus. qPCR assays have also been developed for detection of VSV-G as a means for detecting replication competent virus. In another example, levels of reverse transcriptase activity are measured (e.g. using F-PERT) as a means for detecting replication competent virus (i.e. the presence of replication competent virus is tested using a reverse transcriptase assay such as F-PERT). As a non-limiting example, detection of replication competent virus using F-PERT is described in detail in the examples section below.

Alternative assays, such as protein based assays, may also be used to detect replication competent virus. For example, an ELISA assays for detecting p24 have previously been developed and may be used.

PCR based methods are well known in the art. Appropriate reagents and methodology may readily be identified by a person of skill in the art. Similarly, protein detection methods (e.g. ELISA) are also well known in the art. Appropriate reagents and methodology may readily be identified by a person of skill in the art.

The methods described above detect the presence of replication competent virus in the sample. As used herein, “detecting” refers to indicating the presence of replication competent virus in the sample. Replication competent virus is detected when the methods indicate the presence of e.g. a viral gene, a viral protein, and/or viral activity e.g. reverse transcriptase activity in the sample with a given value. The given value is typically compared to a reference value, and/or a corresponding value generated from a positive control, and/or a corresponding value generated from a negative control. Typically, a given value that is at or above the reference value (a threshold value above which replication competent virus is present) is deemed to indicate the presence of replication competent virus in the test sample. Conversely, a given value that is below the reference value is deemed to indicate that the sample is free from replication competent virus. Appropriate reference values and controls (both positive and negative) are well known in the art.

### General Definitions

Several general definitions are provided below.

### Culture of Production Cells

Production cells, either packaging or producer cell lines or those transiently transfected with the viral vector encoding components are cultured to increase cell and virus numbers and/or virus titres. Culturing a cell is performed to enable it to metabolize, and/or grow and/or divide and/or produce viral vectors of interest. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture multi-well plates, dishes, roller bottles, wave bags or in bioreactors, using batch, fed-batch, continuous systems and the like. In order to achieve large scale production of viral vector through cell culture it is preferred in the art to have cells capable of growing in suspension.

### Nucleic Acid

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

### Vector

A vector is a tool that allows or facilitates the transfer of an entity from one environment to another. By way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleic acid/nucleotide of interest (NOI) to maintain the NOI and its expression within the target cell. Alternatively, the vector may facilitate the replication of the vector through expression of the NOI in a transient system. The vector may serve the purposes of maintaining the heterologous nucleic acid (DNA or RNA) within the cell, or facilitating the replication of the vector comprising a segment of DNA or RNA or the expression of the protein encoded by a segment of nucleic acid. The vector may facilitate the integration of the nucleic acid/nucleotide of interest (NOI) to maintain the NOI and its expression within the target cell. Alternatively, the vector may facilitate the replication of the vector through expression of the NOI in a transient system.

In the context of the methods described herein, the vectors of interest are viral vectors, in particular retroviral vectors. A viral vector may also be called a vector, vector virion or vector particle. The vectors may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell.

The vector may be an expression vector. Expression vectors as described herein comprise regions of nucleic acid containing sequences capable of being transcribed. Thus, sequences encoding mRNA, tRNA and rRNA are included within this definition. Preferably, an expression vector comprises a polynucleotide of the invention operably linked to a control sequence that is capable of providing for the expression of the coding sequence by the target cell.

### Retroviral Vectors

Retroviral vectors may be derived from or may be derivable from any suitable retrovirus. A large number of different retroviruses have been identified. Examples include: murine leukemia virus (MLV), human T-cell leukemia virus (HTLV), mouse mammary tumour virus (MMTV), Rous sarcoma virus (RSV), Fujinami sarcoma virus (FuSV), Moloney murine leukemia virus (Mo MLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A detailed list of retroviruses may be found in Coffin et al. (1997) “Retroviruses”, Cold Spring Harbour Laboratory Press Eds: JM Coffin, SM Hughes, HE Varmus pp 758-763.

Retroviruses may be broadly divided into two categories, namely “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al (1997) ibid.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components -these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical retroviral vector, at least part of one or more protein coding regions essential for replication may be removed from the virus; for example, gag/pol and env may be absent or not functional. This makes the viral vector replication-defective.

Lentiviruses are part of a larger group of retroviruses. A detailed list of lentiviruses may be found in Coffin et al (1997) “Retroviruses” Cold Spring Harbour Laboratory Press Eds: JM Coffin, SM Hughes, HE Varmus pp 758-763). A lentiviral vector, as used herein, is a vector which comprises at least one component part derivable from a lentivirus. Preferably, that component part is involved in the biological mechanisms by which the vector infects or transduces target cells and expresses NOI.

In brief, lentiviruses can be divided into primate and non-primate groups. Examples of primate lentiviruses include but are not limited to: the human immunodeficiency virus (HIV e.g. HIV-1 or HIV-2), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). Other examples include a visna lentivirus.

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

### Adenoviral and Adeno-Associated Viral Vectors

Adenoviruses may also be detected using the methods described herein. An adenovirus is a double-stranded, linear DNA virus that does not replicate through an RNA intermediate. There are over 50 different human serotypes of adenovirus divided into 6 subgroups based on their genetic sequence.

Adenoviruses are double-stranded DNA non-enveloped viruses that are capable of in vivo, ex vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons.

Adenoviral vectors are also capable of transducing non-dividing cells. This is very important for diseases, such as cystic fibrosis, in which the affected cells in the lung epithelium have a slow turnover rate. In fact, several trials are underway utilising adenovirus-mediated transfer of cystic fibrosis transporter (CFTR) into the lungs of afflicted adult cystic fibrosis patients.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 1012 transducing units per ml. Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus.

The use of recombinant adeno-associated viral (AAV) and Adenovirus based viral vectors for gene therapy is widespread, and manufacture of the same has been well documented. Typically, AAV-based vectors are produced in mammalian cell lines (e.g. HEK293-based) or through use of the baculovirus/Sf9 insect cell system. AAV vectors can be produced by transient transfection of vector component encoding DNAs, typically together with helper functions from Adenovirus or Herpes Simplex virus (HSV), or by use of cell lines stably expressing AAV vector components. Adenoviral vectors are typically produced in mammalian cell lines that stably express Adenovirus E1 functions (e.g. HEK293-based).

Adenoviral vectors are also typically ‘amplified’ via helper-function-dependent replication through serial rounds of ‘infection’ using the production cell line. An adenoviral vector and production system thereof comprises a polynucleotide comprising all or a portion of an adenovirus genome. It is well known that an adenovirus is, without limitation, an adenovirus derived from Ad2, Ad5, Ad12, and Ad40. An adenoviral vector is typically in the form of DNA encapsulated in an adenovirus coat or adenoviral DNA packaged in another viral or viral-like form (such as herpes simplex, and AAV).

An AAV vector it is commonly understood to be a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. An ‘AAV vector’ also refers to its protein shell or capsid, which provides an efficient vehicle for delivery of vector nucleic acid to the nucleus of target cells. AAV production systems require helper functions which typically refers to AAV-derived coding sequences which can be expressed to provide AAV gene products that, in turn, function in trans for productive AAV replication. As such, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors. It is understood that a AAV helper construct refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and plM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. Nos. 5,139,941 and 6,376,237. In addition, it is common knowledge that the term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

### Herpes Simplex Virus Vectors

Herpes simplex virus (HSV) is an enveloped double-stranded DNA virus that naturally infects neurons. It can accommodate large sections of foreign DNA, which makes it attractive as a vector system, and has been employed as a vector for gene delivery to neurons (Manservigiet et al Open Virol J. (2010) 4:123-156).

The use of HSV in therapeutic procedures requires the strains to be attenuated so that they cannot establish a lytic cycle. In particular, if HSV vectors are used for gene therapy in humans, the polynucleotide should preferably be inserted into an essential gene. This is because if a viral vector encounters a wild-type virus, transfer of a heterologous gene to the wild-type virus could occur by recombination. However, as long as the polynucleotide is inserted into an essential gene, this recombinational transfer would also delete the essential gene in the recipient virus and prevent “escape” of the heterologous gene into the replication competent wild-type virus population.

### Vaccinia Virus Vectors

Methods described herein may also be used to detect the presence of a replication competent vaccinia virus. Vaccinia virus vectors include MVA or NYVAC. Alternatives to vaccinia vectors include avipox vectors such as fowlpox or canarypox known as ALVAC and strains derived therefrom which can infect and express recombinant proteins in human cells but are unable to replicate.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

### Sin Vectors

The vectors for use in the methods of the present invention are preferably used in a self-Inactivating (sin) configuration in which the viral enhancer and promoter sequences have Been deleted. Sin vectors can be generated and transduce non-dividing target cells in vivo, Ex vivo or in vitro with an efficacy similar to that of wild-type vectors. The transcriptional Inactivation of the long terminal repeat (ltr) in the sin provirus should prevent mobilization By replication-competent virus. This should also enable the regulated expression of genes From internal promoters by eliminating any cis-acting effects of the ltr.

By way of example, self-inactivating retroviral vector systems have been constructed by Deleting the transcriptional enhancers or the enhancers and promoter in the u3 region of the 3′ ltr. After a round of vector reverse transcription and integration, these changes are Copied into both the 5′ and the 3′ ltrs producing a transcriptionally inactive provirus. However, any promoter(s) internal to the ltrs in such vectors will still be transcriptionally Active. This strategy has been employed to eliminate effects of the enhancers and Promoters in the viral ltrs on transcription from internally placed genes. Such effects Include increased transcription or suppression of transcription. This strategy can also be Used to eliminate downstream transcription from the 3′ ltr into genomic dna. This is of Particular concern in human gene therapy where it is important to prevent the adventitious Activation of any endogenous oncogene. Yu et al., (1986) pnas 83: 3194-98; marty et al., (1990) biochimie 72: 885-7; naviaux et al., (1996) j. Virol. 70: 5701-5; iwakuma et al., (1999) virol. 261: 120-32; deglon et al., (2000) human gene therapy 11: 179-90. Sin Lentiviral vectors are described in us 6,924,123 and us 7,056,699.

The vectors described herein may be pseudotyped with vsv-g (vesicular stomatitis virus-G). This allows for concentration of the virus to high titre.

### Sequence Identity

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™ . For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: -3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

Aspects of the invention are demonstrated by the following non-limiting examples.

### EXAMPLES

### Example 1: RCLCC Assay - Comparison of T225 Flask-Scale and 24W Plate-Scale Assays

### T225 Flask-Scale Assay

RCLCC assays have previously been performed at T225 flask-scale. 10x T225 flasks are seeded with 1.00 E+07 C8166 cells and 1.00 E+07 end of production cells (EOPCs) in a final volume of 50 ml per flask. Thus, the initial seeding density in the RCLCC assay is 4.00 E+05 cells per ml. In order to meet the FDA RCLCC testing guidance, 10x test article flasks are setup to test a total of 1.00 E+08 EOPCs. Flasks are directly passaged for up to 9 passages and supernatant is harvested from passage 6 onwards.

TABLE 3

RCLCC - T225 Flask Scale Calculations

| | | |
|---|---|---|
| Each flask is seeded with: | 1.00E+07 | C8166 cells |
| | 1.00E+07 | EOPCs |
| | 50 | ml total volume |
| Initial Seeding Density | 4.00E+05 | cells per ml |
| | 10 | test article flasks are prepared |
| | 500 | ml cell suspension tested per assay |
| | 2.00E+08 | total cells tested |

Over the duration of the flask-scale RCLCC assay, the total volume of test article that is processed remains constant over the duration of the assay.

TABLE 4

| P1 | 50 | ml total volume | P6 | 50 | ml total volume |
|---|---|---|---|---|---|
| | 10 | test article flasks are prepared | | 10 | test article flasks are prepared |
| | 500 | ml total cell suspension | | 500 | ml total cell suspension |
| P2 | 50 | ml total volume | P7 | 50 | ml total volume |
| | 10 | test article flasks are prepared | | 10 | test article flasks are prepared |
| | 500 | ml total cell suspension | | 500 | ml total cell suspension |
| P3 | 50 | ml total volume | P8 | 50 | ml total volume |
| | 10 | test article flasks are prepared | | 10 | test article flasks are prepared |
| | 500 | ml total cell suspension | | 500 | ml total cell suspension |
| P4 | 50 | ml total volume | P9 | Harvest 500 ml of filtered supernatant | |
| | 10 | test article flasks are prepared | | | |
| | 500 | ml total cell suspension | | | |
| P5 | 50 | ml total volume | | | |
| | 10 | test article flasks are prepared | | | |
| | 500 | ml total cell suspension | | | |

volumes used in different passages of T225 flask-scale assay

### Plate-Scale RCLCC Assay

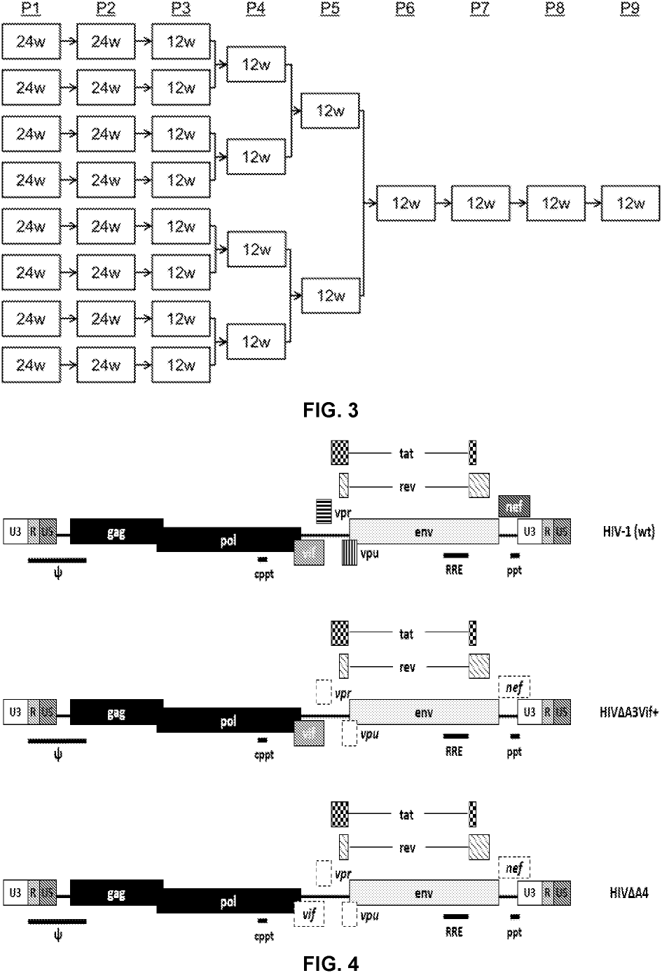

16x 24-well plates were seeded with 2.60 E+05 C8166 cells and 2.60 E+05 end of production cells (EOPCs) in a final volume of 1 ml per well in order to test a total of 1.00 E+08 EOPCs. Plates are directly passaged for up to 9 passages and supernatant was harvested from passage 7 onwards. From passage 3, the 24-well plates were pooled down to a single 24-well plate over subsequent passages (FIG. 3).

TABLE 5

RCLCC - 24 well Plate Scale Calculations

| | | |
|---|---|---|
| Each well is seeded with: | 2.60E+05 | C8166 cells |
| | 2.60E+05 | EOPCs |
| | 1 | ml total volume |
| Initial Seeding Density | 5.20E+05 | cells per ml |
| | 16 | 24 well plates are prepared |
| | 384 | wells total |
| | 384 | ml cell suspension tested per assay |
| | 2.02E+08 | total cells tested |

Over the duration of the plate-scale RCLCC assay, the total volume of test article that is processed is sequentially reduced over the duration of the assay.

TABLE 6

volumes used in different passages of plate-scale assay

| | | | | | |
|---|---|---|---|---|---|
| P1 | 1 | ml total volume per well | P6 | 1 | ml total volume per well |
| | 384 | wells total | | 24 | wells total |
| | 384 | ml total cell suspension | | 24 | ml total cell suspension |
| P2 | 1 | ml total volume per well | P7 | 1 | ml total volume per well |
| | 384 | wells total | | 24 | wells total |
| | 384 | ml total cell suspension | | 24 | ml total cell suspension |
| P3 | 1 | ml total volume per well | P8 | 1 | ml total volume per well |
| | 192 | wells total | | 24 | wells total |
| | 192 | ml total cell suspension | | 24 | ml total cell suspension |
| P4 | 1 | ml total volume per well | P9 | Harvest 24ml of filtered supernatant | |
| | 96 | wells total | | | |
| | 96 | ml total cell suspension | | | |
| P5 | 1 | ml total volume per well | | | |
| | 48 | wells total | | | |
| | 48 | ml total cell suspension | | | |

A comparative study was performed to show that pooling does not compromise assay sensitivity. The data is shown in Table 7A-C below.

The study used three different HIVΔA3Vif+ positive control virus inoculation doses (dose A, B and C) to spike cell culture wells containing cell culture medium and virus permissive cells. The spiked wells were cultured for at least 15 days (with passaging) and then tested for the presence of virus. Two different passaging regimes were tested in parallel: direct passaging (on the left in Table 7) and pooling passaging (on the right in Table 7). Equivalent aliquot volumes (of less than 3 ml) were used throughout the comparative study. Identical results are shown irrespective of whether direct passaging or pooling passaging is used. This demonstrates that pooling does not have an adverse impact on the sensitivity of the assay, even when small aliquot volumes of less than 3 ml are used.

TABLE 7A-C

| Control Batch - Dose A | | | Pooling Batch - Dose A | | |
|---|---|---|---|---|---|
| Expected Rate of Infection: 2-12 wells infected (99.93% probability) | | | | | |
| Sample ID | Well infected (Y/N) | No. Infected Wells | Sample ID | Well infected (Y/N) | No. Infected Wells |
| Spike 1 | - | 9 | Spike 1 | - | 9 |
| Spike 2 | + | | Spike 2 | + | |
| Spike 3 | - | | Spike 3 | - | |
| Spike 4 | + | | Spike 4 | + | |
| Spike 5 | + | | Spike 5 | + | |
| Spike 6 | + | | Spike 6 | + | |
| Spike 7 | + | | Spike 7 | + | |
| Spike 8 | - | | Spike 8 | - | |
| Spike 9 | + | | Spike 9 | + | |
| Spike 10 | + | | Spike 10 | + | |
| Spike 11 | + | | Spike 11 | + | |
| Spike 12 | + | | Spike 12 | + | |
| Spike 12 | - | | Spike 12 | - | |

| | | | | | |
|---|---|---|---|---|---|
| Control Batch - Dose B | | | Pooling Batch - Dose B | | |
| Expected Rate of Infection: 0–8 wells infected (99.97% probability) | | | | | |
| Sample ID | Well infected (Y/N) | No. Infected Wells | Sample ID | Well infected (Y/N) | No. Infected Wells |
| Spike 1 | - | 2 | Spike 1 | - | 2 |
| Spike 2 | + | | Spike 2 | + | |
| Spike 3 | - | | Spike 3 | - | |
| Spike 4 | - | | Spike 4 | - | |
| Spike 5 | - | | Spike 5 | - | |
| Spike 6 | - | | Spike 6 | - | |
| Spike 7 | + | | Spike 7 | + | |
| Spike 8 | - | | Spike 8 | - | |
| Spike 9 | - | | Spike 9 | - | |
| Spike 10 | - | | Spike 10 | - | |
| Spike 11 | - | | Spike 11 | - | |

| | | | | | |
|---|---|---|---|---|---|
| Control Batch - Dose C | | | Pooling Batch - Dose C | | |
| Expected Rate of Infection: 0–5 wells infected (99.97% probability) | | | | | |
| Sample ID | Well infected (Y/N) | No. Infected Wells | Sample ID | Well infected (Y/N) | No. Infected Wells |
| Spike 1 | - | 1 | Spike 1 | - | 1 |
| Spike 2 | - | | Spike 2 | - | |
| Spike 3 | - | | Spike 3 | - | |
| Spike 4 | - | | Spike 4 | - | |
| Spike 5 | + | | Spike 5 | + | |
| Spike 6 | - | | Spike 6 | - | |
| Spike 7 | - | | Spike 7 | - | |
| Spike 8 | - | | Spike 8 | - | |
| Spike 9 | - | | Spike 9 | - | |
| Spike 10 | - | | Spike 10 | - | |
| Spike 11 | - | | Spike 11 | - | |

Table 7A-C: shows the infection rate of the HIVΔA3Vif+ positive control virus assessed at three inoculation doses under two passaging regimes (Control Batch - Direct passaging only; Pooling Batch – Pooling passaging from day 9). All observed rates of infection were as expected in both the Control and Pooling Batches at all three inoculation doses. Likewise all infected wells in the Control Batch were also infected in the Pooling Batch, demonstrating that pooling does not compromise assay sensitivity

### Example 2: Generation of New HIV-1 Positive Controls

Lentiviral vectors for use in gene therapy are typically developed from several vector system components. HIV-based minimal 3^{rd} generation vector systems are devoid of accessory genes vif, vpr, vpu and nef, and the auxiliary gene tat. The standard vector genome comprises a packaging signal (Ψ), the rev response element (RRE), the central polypurine tract (cppt), the internal nucleotide of interest (NOI) expression cassette, typically a post-transcription regulatory element (PRE), the 3′ polypurine tract (ppt) and a self-inactivating (SIN) LTR. The production system employs four core components encoded on separate DNAs: vector genome, gagpol, rev and envelope . Third generation vectors may utilise a wild type or codon-optimised gagpol ORF, but the use of the latter greatly reduces the probability of homologous recombination between vector components that might result in generation of an RCL. Nevertheless, a requirement for clinical release of final vector drug product is to test for the presence of RCL.

One aspect of RCL assay design is to utilise an appropriate positive control virus. Two positive controls were therefore generated: HIVΔA4 and HIVΔA3Vif+ (see FIG. 4).

### Generation of HIVΔA4

Wild type HIV-1 was engineered such that accessory genes vif, vpr, vpu and nef were functionally deleted to generate HIVΔA4, thus modelling a putative RCL that might arise from the minimal vector system.

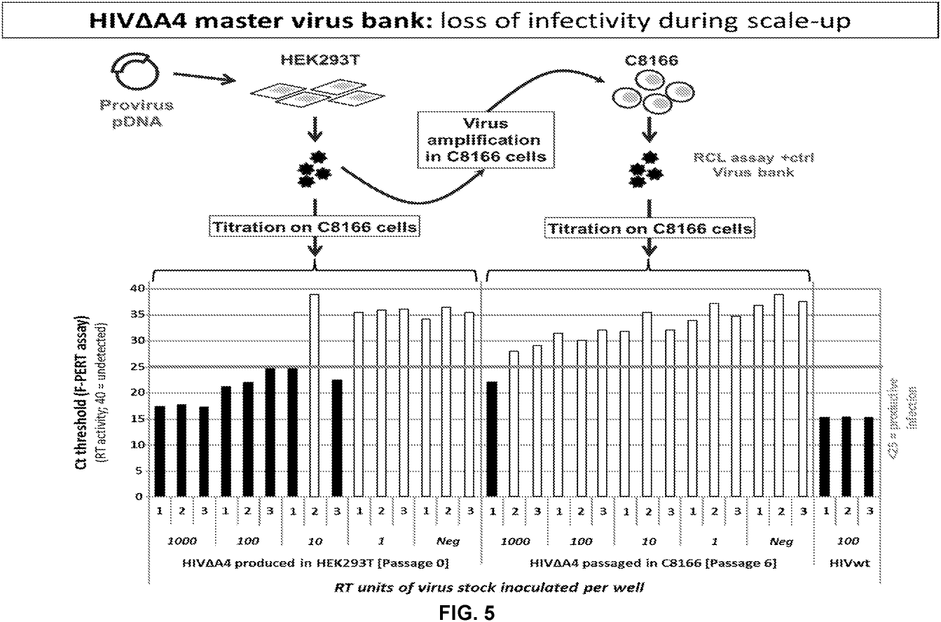

The C8166-45 cell line was derived by T-cell immortalisation through HTLV-1 tax1 expression, and are highly permissive for HIV-1 infection. These cells are therefore typically used as an RCL assay amplification cell line using HIV-1-based positive controls. However, other (less) permissive cells that have been evaluated for sensitivity to infection by wild type or attenuated HIV-1 include CEM-SS, MT4, Molt4, Molt4.8, PM1, H9, Jurkat and SupT1 cells. Initially, a large master virus bank of HIVΔA4 was generated by transfecting HEK293T cells with proviral DNA, amplified the virus through C8166-45 cells and then quantified the physical titre of the bank by Fluorescence Product-Enhanced Reverse Transcriptase (F-PERT) assay. Using this data the infectious titre of the bank was determined by serial dilution-infection of C8166-45 cells at 48-well scale. However, the master virus bank was ~1000-fold less infectious compared to the HEK293T-made starting virus (FIG. 5).

An F-PERT assay is a well described protocol and would be known to a person skilled in the art. For example, samples may be disrupted/lysed using a lysis buffer/solution and analysed neat via F-PERT qPCR. A F-PERT mastermix may contain MS2 RNA and primers and a probe specific to MS2. Levels of reverse transcriptase activity are measured relative to a RT standard with known activity levels.

### Generation of HIVΔA3Vif+

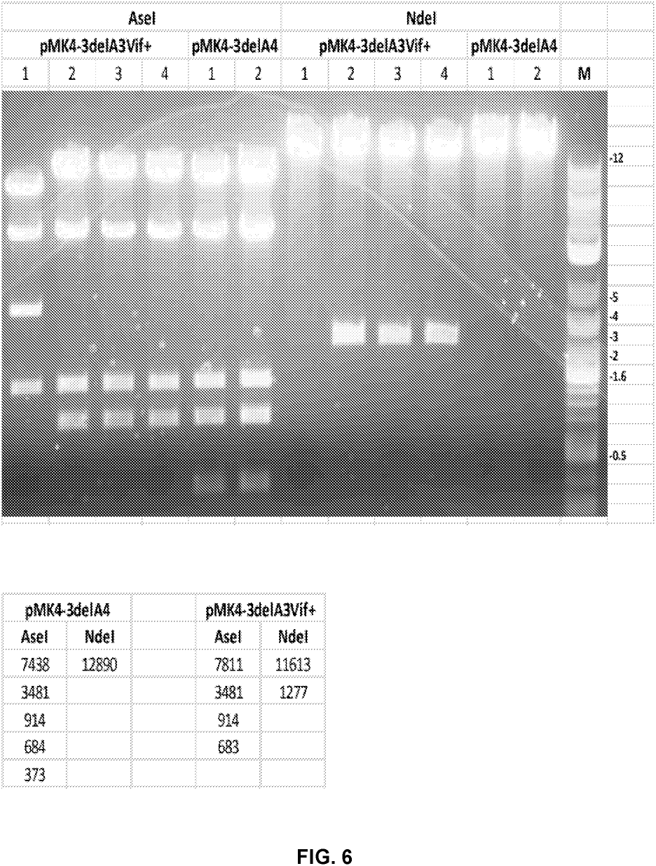

A synthetic plasmid (pVif Repair) was made by GeneArt and a Sbfl-EcoRl fragment inserted into pMK4-3ΔA4 (Miniprep H10). Clone DNA from new minipreps were digested to screen for pMK4-3ΔA3Vif+. An additional Ndel site is present and an Asel site is lost in pMK4-3ΔA3Vif+ (FIG. 6).

DNA from clones 3 and 4 were pooled and used to generate a virus stock of HIVΔA3Vif+.

### Production of HIVΔA3Vif+ Master Virus Stock

### Cell Seeding

HEK293T cells were taken from the routine GLP passage stock. A T150 flask was seeded with 9.2×106 cells/26.3 mLand a 10 cm2 plate seeded with 3.5×106 cells/10 mL, and cultures incubated overnight at 37° C.

### Transfection of Cells With Proviral DNA

Cultures appeared healthy and were transfected with proviral DNA as below.

TABLE 8

transfection protocol

| | a | | b | c | |
|---|---|---|---|---|---|
| ug/ul: | 0.385 | 0.3 | | | |
| | pMK4-3ΔA4 (cl1) | pMK4-3ΔA3Vif+ (cl3, cl4) | OPTiM | LK/OPTiM | Vol/vessel |
| TXN1 | 15.6 | | 324.4 | 340 | 680 |
| TXN2 | | 52.7 | 842.3 | 895 | 1790 |
| | | | | d | |
| | | | L2K OPTiM | 100 1260 | |

### Protocol

Add DNA (a) to OPTiMem (b) in 5 mL bijou tubes - mix
Add Lipofectamine to OPTiMem (d) in a bijou tube, mix gently - incubated for 5mins
Add L2K/OPTiM (c) dropwise to a+b mixes - swirl thoroughly to mix
Incubate transfection mixes for 25 mins at room tempt
Add TXN mix dropwise to the appropriately labelled cultures - swirl to mix

### Cultures Were Incubated Overnight at 37° C.

### Inductions

Sodium butyrate was added to the cultures to a final culture of 10mM for 6 hours, before 53 mL and 10 mL of fresh media was added to the 10 cm2 plate (TXN1) and T150 flask (TXN2), respectively. Cultures were incubated for ~20 hours at 37° C.

### Virus Harvest

Supernatant from the two cultures were harvested and filtered through a 0.2 µm filter. Fifteen 0.6 mL aliquots of HIVΔA4 and 92x 0.5 mL aliquots of HIVΔA3Vif+ were made in cryovials, and stored at -80° C.

Production of HIVΔA3Vif+ was successful. This master virus bank is now referred to as HIVΔA3Vif+.

### Testing of Infectivity of Wt HIV, HIVΔA3Vif+ and HIVΔA4 in C8166 Cultures

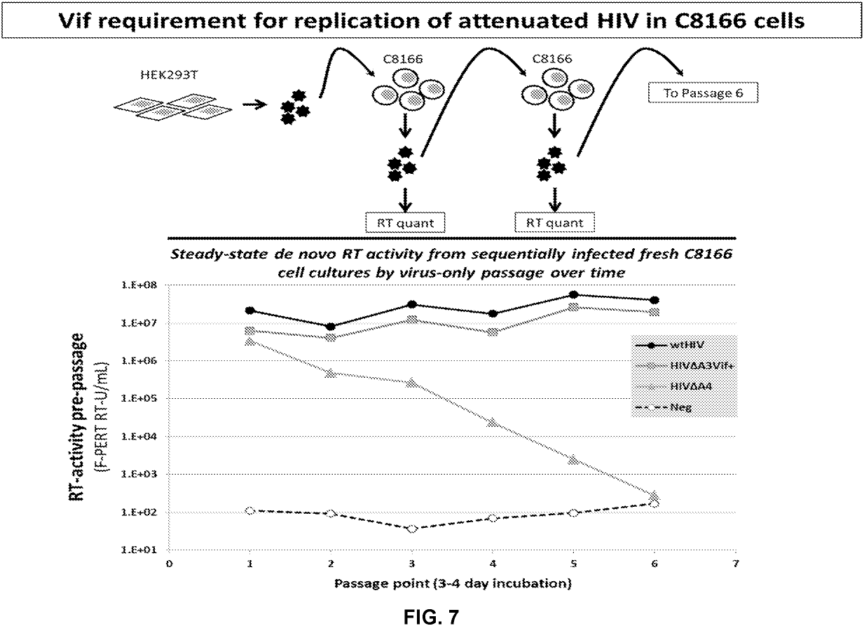

C8166-45 cells have previously been reported to be semi-permissive for vif-deficient HIV-1. The infectivity of wt HIV, HIVΔA3Vif+ and HIVΔA4 was compared by direct, virus-only passage in C8166-45 cultures over 4 weeks. Infections were initiated at MOI ~0.1, incubated 3-4 days before F-PERT analysis, and inoculation of new cultures with 0.1mL of virus-containing supernatant (FIG. 7).

Both wildtype HIV and HIVΔA3Vif+ were capable of serially infecting C8166 cells but HIVΔA4, which is a vif-deficient attenuated HIV virus, became non-infectious over direct passages. This suggested that C8166 cells are semi-permissive for vif-deficient HIV-1.

The reader’s attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### SEQUENCES

SEQ ID NO:1 - HIVΔA3Vif+

TGGAAGGGCTAATTTGGTCCCAAAAAAGACAAGAGATCCTTGATCTGTGG
ATCTACCACACACAAGGCTACTTCCCTGATTGGCAGAACTACACACCAGG
GCCAGGGATCAGATATCCACTGACCTTTGGATGGTGCTTCAAGTTAGTAC
CAGTTGAACCAGAGCAAGTAGAAGAGGCCAATGAAGGAGAGAACAACAGC
TTGTTACACCCTATGAGCCAGCATGGGATGGAGGACCCGGAGGGAGAAGT
ATTAGTGTGGAAGTTTGACAGCCTCCTAGCATTTCGTCACATGGCCCGAG
AGCTGCATCCGGAGTACTACAAAGACTGCTGACATCGAGCTTTCTACAAG
GGACTTTCCGCTGGGGACTTTCCAGGGAGGTGTGGCCTGGGCGGGACTGG
GGAGTGGCGAGCCCTCAGATGCTACATATAAGCAGCTGCTTTTTGCCTGT
ACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTCA
AAGTAGTGTGTGCCCGTCTGTTGTGTGACTCTGGTAACTAGAGATCCCTC
AGACCCTTTTAGTCAGTGTGGAAAATCTCTAGCAGTGGCGCCCGAACAGG
GACTTGAAAGCGAAAGTAAAGCCAGAGGAGATCTCTCGACGCAGGACTCG
GCTTGCTGAAGCGCGCACGGCAAGAGGCGAGGGGCGGCGACTGGTGAGTA
CGCCAAAAATTTTGACTAGCGGAGGCTAGAAGGAGAGAGATGGGTGCGAG
AGCGTCGGTATTAAGCGGGGGAGAATTAGATAAATGGGAAAAAATTCGGT
TAAGGCCAGGGGGAAAGAAACAATATAAACTAAAACATATAGTATGGGCA
AGCAGGGAGCTAGAACGATTCGCAGTTAATCCTGGCCTTTTAGAGACATC
AGAAGGCTGTAGACAAATACTGGGACAGCTACAACCATCCCTTCAGACAG
GATCAGAAGAACTTAGATCATTATATAATACAATAGCAGTCCTCTATTGT
GTGCATCAAAGGATAGATGTAAAAGACACCAAGGAAGCCTTAGATAAGAT
AGAGGAAGAGCAAAACAAAAGTAAGAAAAAGGCACAGCAAGCAGCAGCTG
ACACAGGAAACAACAGCCAGGTCAGCCAAAATTACCCTATAGTGCAGAAC
CTCCAGGGGCAAATGGTACATCAGGCCATATCACCTAGAACTTTAAATGC
ATGGGTAAAAGTAGTAGAAGAGAAGGCTTTCAGCCCAGAAGTAATACCCA
TGTTTTCAGCATTATCAGAAGGAGCCACCCCACAAGATTTAAATACCATG
CTAAACACAGTGGGGGGACATCAAGCAGCCATGCAAATGTTAAAAGAGAC
CATCAATGAGGAAGCTGCAGAATGGGATAGATTGCATCCAGTGCATGCAG
GGCCTATTGCACCAGGCCAGATGAGAGAACCAAGGGGAAGTGACATAGCA
GGAACTACTAGTACCCTTCAGGAACAAATAGGATGGATGACACATAATCC
ACCTATCCCAGTAGGAGAAATCTATAAAAGATGGATAATCCTGGGATTAA
ATAAAATAGTAAGAATGTATAGCCCTACCAGCATTCTGGACATAAGACAA
GGACCAAAGGAACCCTTTAGAGACTATGTAGACCGATTCTATAAAACTCT
AAGAGCCGAGCAAGCTTCACAAGAGGTAAAAAATTGGATGACAGAAACCT
TGTTGGTCCAAAATGCGAACCCAGATTGTAAGACTATTTTAAAAGCATTG
GGACCAGGAGCGACACTAGAAGAAATGATGACAGCATGTCAGGGAGTGGG
GGGACCCGGCCATAAAGCAAGAGTTTTGGCTGAAGCAATGAGCCAAGTAA
CAAATCCAGCTACCATAATGATACAGAAAGGCAATTTTAGGAACCAAAGA
AAGACTGTTAAGTGTTTCAATTGTGGCAAAGAAGGGCACATAGCCAAAAA
TTGCAGGGCCCCTAGGAAAAAGGGCTGTTGGAAATGTGGAAAGGAAGGAC
ACCAAATGAAAGATTGTACTGAGAGACAGGCTAATTTTTTAGGGAAGATC
TGGCCTTCCCACAAGGGAAGGCCAGGGAATTTTCTTCAGAGCAGACCAGA
GCCAACAGCCCCACCAGAAGAGAGCTTCAGGTTTGGGGAAGAGACAACAA
CTCCCTCTCAGAAGCAGGAGCCGATAGACAAGGAACTGTATCCTTTAGCT
TCCCTCAGATCACTCTTTGGCAGCGACCCCTCGTCACAATAAAGATAGGG
GGGCAATTAAAGGAAGCTCTATTAGATACAGGAGCAGATGATACAGTATT
AGAAGAAATGAATTTGCCAGGAAGATGGAAACCAAAAATGATAGGGGGAA
TTGGAGGTTTTATCAAAGTAAGACAGTATGATCAGATACTCATAGAAATC
TGCGGACATAAAGCTATAGGTACAGTATTAGTAGGACCTACACCTGTCAA
CATAATTGGAAGAAATCTGTTGACTCAGATTGGCTGCACTTTAAATTTTC
CCATTAGTCCTATTGAGACTGTACCAGTAAAATTAAAGCCAGGAATGGAT
GGCCCAAAAGTTAAACAATGGCCATTGACAGAAGAAAAAATAAAAGCATT
AGTAGAAATTTGTACAGAAATGGAAAAGGAAGGAAAAATTTCAAAAATTG
GGCCTGAAAATCCATACAATACTCCAGTATTTGCCATAAAGAAAAAAGAC
AGTACTAAATGGAGAAAATTAGTAGATTTCAGAGAACTTAATAAGAGAAC
TCAAGATTTCTGGGAAGTTCAATTAGGAATACCACATCCTGCAGGGTTAA
AACAGAAAAAATCAGTAACAGTACTGGATGTGGGCGATGCATATTTTTCA
GTTCCCTTAGATAAAGACTTCAGGAAGTATACTGCATTTACCATACCTAG
TATAAACAATGAGACACCAGGGATTAGATATCAGTACAATGTGCTTCCAC
AGGGATGGAAAGGATCACCAGCAATATTCCAGTGTAGCATGACAAAAATC
TTAGAGCCTTTTAGAAAACAAAATCCAGACATAGTCATCTATCAATACAT
GGATGATTTGTATGTAGGATCTGACTTAGAAATAGGGCAGCATAGAACAA
AAATAGAGGAACTGAGACAACATCTGTTGAGGTGGGGATTTACCACACCA
GACAAAAAACATCAGAAAGAACCTCCATTCCTTTGGATGGGTTATGAACT
CCATCCTGATAAATGGACAGTACAGCCTATAGTGCTGCCAGAAAAGGACA
GCTGGACTGTCAATGACATACAGAAATTAGTGGGAAAATTGAATTGGGCA
AGTCAGATTTATGCAGGGATTAAAGTAAGGCAATTATGTAAACTTCTTAG
GGGAACCAAAGCACTAACAGAAGTAGTACCACTAACAGAAGAAGCAGAGC
TAGAACTGGCAGAAAACAGGGAGATTCTAAAAGAACCGGTACATGGAGTG
TATTATGACCCATCAAAAGACTTAATAGCAGAAATACAGAAGCAGGGGCA
AGGCCAATGGACATATCAAATTTATCAAGAGCCATTTAAAAATCTGAAAA
CAGGAAAGTATGCAAGAATGAAGGGTGCCCACACTAATGATGTGAAACAA
TTAACAGAGGCAGTACAAAAAATAGCCACAGAAAGCATAGTAATATGGGG
AAAGACTCCTAAATTTAAATTACCCATACAAAAGGAAACATGGGAAGCAT
GGTGGACAGAGTATTGGCAAGCCACCTGGATTCCTGAGTGGGAGTTTGTC
AATACCCCTCCCTTAGTGAAGTTATGGTACCAGTTAGAGAAAGAACCCAT
AATAGGAGCAGAAACTTTCTATGTAGATGGGGCAGCCAATAGGGAAACTA
AATTAGGAAAAGCAGGATATGTAACTGACAGAGGAAGACAAAAAGTTGTC
CCCCTAACGGACACAACAAATCAGAAGACTGAGTTACAAGCAATTCATCT
AGCTTTGCAGGATTCGGGATTAGAAGTAAACATAGTGACAGACTCACAAT
ATGCATTGGGAATCATTCAAGCACAACCAGATAAGAGTGAATCAGAGTTA
GTCAGTCAAATAATAGAGCAGTTAATAAAAAAGGAAAAAGTCTACCTGGC
ATGGGTACCAGCACACAAAGGAATTGGAGGAAATGAACAAGTAGATAAAT
TGGTCAGTGCTGGAATCAGGAAAGTACTATTTTTAGATGGAATAGATAAG
GCCCAAGAAGAACATGAGAAATATCACAGTAATTGGAGAGCAATGGCTAG
TGATTTTAACCTACCACCTGTAGTAGCAAAAGAAATAGTAGCCAGCTGTG
ATAAATGTCAGCTAAAAGGGGAAGCCATGCATGGACAAGTAGACTGTAGC
CCAGGAATATGGCAGCTAGATTGTACACATTTAGAAGGAAAAGTTATCTT
GGTAGCAGTTCATGTAGCCAGTGGATATATAGAAGCAGAAGTAATTCCAG
CAGAGACAGGGCAAGAAACAGCATACTTCCTCTTAAAATTAGCAGGAAGA
TGGCCAGTAAAAACAGTACATACAGACAATGGCAGCAATTTCACCAGTAC
TACAGTTAAGGCCGCCTGTTGGTGGGCGGGGATCAAGCAGGAATTTGGCA
TTCCCTACAATCCCCAAAGTCAAGGAGTAATAGAATCTATGAATAAAGAA
TTAAAGAAAATTATAGGACAGGTAAGAGATCAGGCTGAACATCTTAAGAC
AGCAGTACAAATGGCAGTATTCATCCACAATTTTAAAAGAAAAGGGGGGA
TTGGGGGGTACAGTGCAGGGGAAAGAATAGTAGACATAATAGCAACAGAC
ATACAAACTAAAGAATTACAAAAACAAATTACAAAAATTCAAAATTTTCG
GGTTTATTACAGGGACAGCAGAGATCCAGTTTGGAAAGGACCAGCAAAGC
TCCTCTGGAAAGGTGAAGGGGCAGTAGTAATACAAGATAATAGTGACATA
AAAGTAGTGCCAAGAAGAAAAGCAAAGATCATCAGGGATTACGGAAAACA
GATGGCAGGTGACGATTGTGTGGCAAGTAGACAGGACGAGGATTAACACA
TGGAAAAGATTAGTAAAACACCATTAATATATTTCAAGGAAAGCTAAGGA
CTGGTTTTATAGACATCACTATGAAAGTACTAATCCAAAAATAAGTTCAG
AAGTACACATCCCACTAGGGGATGCTAAATTAGTAATAACAACATATTGG
GGTCTGCATACAGGAGAAAGAGACTGGCATTTGGGTCAGGGAGTCTCCAT
AGAATGGAGGAAAAAGAGATATAGCACACAAGTAGACCCTGACCTAGCAG
ACCAACTAATTCATCTGCACTATTTTGATTGTTTTTCAGAATCTGCTATA
AGAAATACCATATTAGGACGTATAGTTAGTTAAAGGTGTGAATATCAAGC
AGGACATAACAAGGTAGGATCTCTACAGTACTTGGCACTAGCAGCATTAA
TAAAACCAAAACAGATAAAGCCACCTTTGCCTAGTGTTAGGAAACTGACA
GAGGACAGCCCGAACAAGCCCCAGAAGACCAAGGGCCACAGAGGGAGCCA
TACAATGAATGGACACTAGAGCTTTTAGAGGAACTTAAGAGTGAAGCTGT
TAGACATTTTCCTAGGATATGGCTCCATAACTTAGGACAACATATCTATG
AAACTTACGGGGATACTTGGGCAGGAGTGGAATAAATAATAAGAATTCTG
CAACAACTGCTGTTTATCCATTTCAGAATTGGGTGTCGACATAGCAGAAT
AGGCGTTACTCGACAGAGGAGAGCAAGAAATGGAGCCAGTAGATCCTAGA
CTAGAGCCCTGGAAGCATCCAGGAAGTCAGCCTAAAACTGCTTGTACCAA
TTGCTATTGTAAAAAGTGTTGCTTTCATTGCCAAGTTTGTTTCATGACAA
AAGCCTTAGGCATCTCCTATGGCAGGAAGAAGCGGAGACAGCGACGAAGA
GCTCATCAGAACAGTCAGACTCATCAAGCTTCTCTATCAAAGCAGTAAGT
AGTACATGTACCCCAACCTATAATAGTAGCAATAGTAGCATTAGTAGTAG
CAATAATAATAGCAATAGTTGTGTGGTCCATAGTAATCATAGAATATAGG
AAAATATTAAGACAAAGAAAAATAGACTAATTAATTGATAGACTAATAGA
AAGAGCAGAAGACAGTGGCAATGAGAGTGAAGGAGAAGTATCAGCACTTG
TGGAGATGGGGGTGGAAATGGGGCACCATGCTCCTTGGGATATTGATGAT
CTGTAGTGCTACAGAAAAATTGTGGGTCACAGTCTATTATGGGGTACCTG
TGTGGAAGGAAGCAACCACCACTCTATTTTGTGCATCAGATGCTAAAGCA
TATGATACAGAGGTACATAATGTTTGGGCCACACATGCCTGTGTACCCAC
AGACCCCAACCCACAAGAAGTAGTATTGGTAAATGTGACAGAAAATTTTA
ACATGTGGAAAAATGACATGGTAGAACAGATGCATGAGGATATAATCAGT
TTATGGGATCAAAGCCTAAAGCCATGTGTAAAATTAACCCCACTCTGTGT
TAGTTTAAAGTGCACTGATTTGAAGAATGATACTAATACCAATAGTAGTA
GCGGGAGAATGATAATGGAGAAAGGAGAGATAAAAAACTGCTCTTTCAAT
ATCAGCACAAGCATAAGAGATAAGGTGCAGAAAGAATATGCATTCTTTTA
TAAACTTGATATAGTACCAATAGATAATACCAGCTATAGGTTGATAAGTT
GTAACACCTCAGTCATTACACAGGCCTGTCCAAAGGTATCCTTTGAGCCA
ATTCCCATACATTATTGTGCCCCGGCTGGTTTTGCGATTCTAAAATGTAA
TAATAAGACGTTCAATGGAACAGGACCATGTACAAATGTCAGCACAGTAC
AATGTACACATGGAATCAGGCCAGTAGTATCAACTCAACTGCTGTTAAAT
GGCAGTCTAGCAGAAGAAGATGTAGTAATTAGATCTGCCAATTTCACAGA
CAATGCTAAAACCATAATAGTACAGCTGAACACATCTGTAGAAATTAATT
GTACAAGACCCAACAACAATACAAGAAAAAGTATCCGTATCCAGAGGGGA
CCAGGGAGAGCATTTGTTACAATAGGAAAAATAGGAAATATGAGACAAGC
ACATTGTAACATTAGTAGAGCAAAATGGAATGCCACTTTAAAACAGATAG
CTAGCAAATTAAGAGAACAATTTGGAAATAATAAAACAATAATCTTTAAG
CAATCCTCAGGAGGGGACCCAGAAATTGTAACGCACAGTTTTAATTGTGG
AGGGGAATTTTTCTACTGTAATTCAACACAACTGTTTAATAGTACTTGGT
TTAATAGTACTTGGAGTACTGAAGGGTCAAATAACACTGAAGGAAGTGAC
ACAATCACACTCCCATGCAGAATAAAACAATTTATAAACATGTGGCAGGA
AGTAGGAAAAGCAATGTATGCCCCTCCCATCAGTGGACAAATTAGATGTT
CATCAAATATTACTGGGCTGCTATTAACAAGAGATGGTGGTAATAACAAC
AATGGGTCCGAGATCTTCAGACCTGGAGGAGGCGATATGAGGGACAATTG
GAGAAGTGAATTATATAAATATAAAGTAGTAAAAATTGAACCATTAGGAG
TAGCACCCACCAAGGCAAAGAGAAGAGTGGTGCAGAGAGAAAAAAGAGCA
GTGGGAATAGGAGCTTTGTTCCTTGGGTTCTTGGGAGCAGCAGGAAGCAC
TATGGGCGCAGCGTCAATGACGCTGACGGTACAGGCCAGACAATTATTGT
CTGATATAGTGCAGCAGCAGAACAATTTGCTGAGGGCTATTGAGGCGCAA
CAGCATCTGTTGCAACTCACAGTCTGGGGCATCAAACAGCTCCAGGCAAG
AATCCTGGCTGTGGAAAGATACCTAAAGGATCAACAGCTCCTGGGGATTT
GGGGTTGCTCTGGAAAACTCATTTGCACCACTGCTGTGCCTTGGAATGCT
AGTTGGAGTAATAAATCTCTGGAACAGATTTGGAATAACATGACCTGGAT
GGAGTGGGACAGAGAAATTAACAATTACACAAGCTTAATACACTCCTTAA
TTGAAGAATCGCAAAACCAGCAAGAAAAGAATGAACAAGAATTATTGGAA
TTAGATAAATGGGCAAGTTTGTGGAATTGGTTTAACATAACAAATTGGCT
GTGGTATATAAAATTATTCATAATGATAGTAGGAGGCTTGGTAGGTTTAA
GAATAGTTTTTGCTGTACTTTCTATAGTGAATAGAGTTAGGCAGGGATAT
TCACCATTATCGTTTCAGACCCACCTCCCAATCCCGAGGGGACCCGACAG
GCCCGAAGGAATAGAAGAAGAAGGTGGAGAGAGAGACAGAGACAGATCCA
TTCGATTAGTGAACGGATCCTTAGCACTTATCTGGGACGATCTGCGGAGC
CTGTGCCTCTTCAGCTACCACCGCTTGAGAGACTTACTCTTGATTGTAAC
GAGGATTGTGGAACTTCTGGGACGCAGGGGGTGGGAAGCCCTCAAATATT
GGTGGAATCTCCTACAGTATTGGAGTCAGGAACTAAAGAATAGTGCTGTT
AACTTGCTCAATGCCACAGCCATAGCAGTAGCTGAGGGGACAGATAGGGT
TATAGAAGTATTACAAGCAGCTTATAGAGCTATTCGCCACATACCTAGAA
GAATAAGACAGGGCTTGGAAAGGATTTTGCTATAAGCCCGGTGGCAAGTG
GTCAAAAAGTAGTGTGATTGGATGGCCTGCTGTAAGGGAAAGATAAAGAC
GAGCTGAGCCAGCAGCAGATGGGGTGGGAGCAGTATCTCGAGACCTAGAA
AAACATGGAGCAATCACAAGTAGCAATACAGCAGCTAACAATGCTGCTTG
TGCCTGGCTAGAAGCACAAGAGGAGGAAGAGGTGGGTTTTCCAGTCACAC
CTCAGGTACCTTTAAGACCAATGACTTACAAGGCAGCTGTAGATCTTAGC
CACTTTTTAAAAGAAAAGGGGGGACTGGAAGGGCTAATTCACTCCCAAAG
AAGACAAGATATCCTTGATCTGTGGATCTACCACACACAATAATACTTCC
CTGATTGGCAGAACTACACACCAGGGCCAGGGGTCAGATATCCACTGACC
TTTGGATGGTGCTACAAGCTAGTACCAGTTGAGCCAGATAAGGTAGAAGA
GGCCAATAAAGGAGAGAACACCAGCTTGTTACACCCTGTGAGCCTGCATG
GAATGGATGACCCTGAGAGAGAAGTGTTAGAGTGGAGGTTTGACAGCCGC
CTAGCATTTCATCACGTGGCCCGAGAGCTGCATCCGGAGTACTTCAAGAA
CTGCTGACATCGAGCTTGCTACAAGGGACTTTCCGCTGGGGACTTTCCAG
GGAGGCGTGGCCTGGGCGGGACTGGGGAGTGGCGAGCCCTCAGATGCTGC
ATATAAGCAGCTGCTTTTTGCCTGTACTGGGTCTCTCTGGTTAGACCAGA
TCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTG
TGACTCTGGTAACTAGAGATCCCTCAGACC

SEQ ID NO: 2 HIVΔA4

TGGAAGGGCTAATTTGGTCCCAAAAAAGACAAGAGATCCTTGATCTGTGG
ATCTACCACACACAAGGCTACTTCCCTGATTGGCAGAACTACACACCAGG
GCCAGGGATCAGATATCCACTGACCTTTGGATGGTGCTTCAAGTTAGTAC
CAGTTGAACCAGAGCAAGTAGAAGAGGCCAATGAAGGAGAGAACAACAGC
TTGTTACACCCTATGAGCCAGCATGGGATGGAGGACCCGGAGGGAGAAGT
ATTAGTGTGGAAGTTTGACAGCCTCCTAGCATTTCGTCACATGGCCCGAG
AGCTGCATCCGGAGTACTACAAAGACTGCTGACATCGAGCTTTCTACAAG
GGACTTTCCGCTGGGGACTTTCCAGGGAGGTGTGGCCTGGGCGGGACTGG
GGAGTGGCGAGCCCTCAGATGCTACATATAAGCAGCTGCTTTTTGCCTGT
ACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTCA
AAGTAGTGTGTGCCCGTCTGTTGTGTGACTCTGGTAACTAGAGATCCCTC
AGACCCTTTTAGTCAGTGTGGAAAATCTCTAGCAGTGGCGCCCGAACAGG
GACTTGAAAGCGAAAGTAAAGCCAGAGGAGATCTCTCGACGCAGGACTCG
GCTTGCTGAAGCGCGCACGGCAAGAGGCGAGGGGCGGCGACTGGTGAGTA
CGCCAAAAATTTTGACTAGCGGAGGCTAGAAGGAGAGAGATGGGTGCGAG
AGCGTCGGTATTAAGCGGGGGAGAATTAGATAAATGGGAAAAAATTCGGT
TAAGGCCAGGGGGAAAGAAACAATATAAACTAAAACATATAGTATGGGCA
AGCAGGGAGCTAGAACGATTCGCAGTTAATCCTGGCCTTTTAGAGACATC
AGAAGGCTGTAGACAAATACTGGGACAGCTACAACCATCCCTTCAGACAG
GATCAGAAGAACTTAGATCATTATATAATACAATAGCAGTCCTCTATTGT
GTGCATCAAAGGATAGATGTAAAAGACACCAAGGAAGCCTTAGATAAGAT
AGAGGAAGAGCAAAACAAAAGTAAGAAAAAGGCACAGCAAGCAGCAGCTG
ACACAGGAAACAACAGCCAGGTCAGCCAAAATTACCCTATAGTGCAGAAC
CTCCAGGGGCAAATGGTACATCAGGCCATATCACCTAGAACTTTAAATGC
ATGGGTAAAAGTAGTAGAAGAGAAGGCTTTCAGCCCAGAAGTAATACCCA
TGTTTTCAGCATTATCAGAAGGAGCCACCCCACAAGATTTAAATACCATG
CTAAACACAGTGGGGGGACATCAAGCAGCCATGCAAATGTTAAAAGAGAC
CATCAATGAGGAAGCTGCAGAATGGGATAGATTGCATCCAGTGCATGCAG
GGCCTATTGCACCAGGCCAGATGAGAGAACCAAGGGGAAGTGACATAGCA
GGAACTACTAGTACCCTTCAGGAACAAATAGGATGGATGACACATAATCC
ACCTATCCCAGTAGGAGAAATCTATAAAAGATGGATAATCCTGGGATTAA
ATAAAATAGTAAGAATGTATAGCCCTACCAGCATTCTGGACATAAGACAA
GGACCAAAGGAACCCTTTAGAGACTATGTAGACCGATTCTATAAAACTCT
AAGAGCCGAGCAAGCTTCACAAGAGGTAAAAAATTGGATGACAGAAACCT
TGTTGGTCCAAAATGCGAACCCAGATTGTAAGACTATTTTAAAAGCATTG
GGACCAGGAGCGACACTAGAAGAAATGATGACAGCATGTCAGGGAGTGGG
GGGACCCGGCCATAAAGCAAGAGTTTTGGCTGAAGCAATGAGCCAAGTAA
CAAATCCAGCTACCATAATGATACAGAAAGGCAATTTTAGGAACCAAAGA
AAGACTGTTAAGTGTTTCAATTGTGGCAAAGAAGGGCACATAGCCAAAAA
TTGCAGGGCCCCTAGGAAAAAGGGCTGTTGGAAATGTGGAAAGGAAGGAC
ACCAAATGAAAGATTGTACTGAGAGACAGGCTAATTTTTTAGGGAAGATC
TGGCCTTCCCACAAGGGAAGGCCAGGGAATTTTCTTCAGAGCAGACCAGA
GCCAACAGCCCCACCAGAAGAGAGCTTCAGGTTTGGGGAAGAGACAACAA
CTCCCTCTCAGAAGCAGGAGCCGATAGACAAGGAACTGTATCCTTTAGCT
TCCCTCAGATCACTCTTTGGCAGCGACCCCTCGTCACAATAAAGATAGGG
GGGCAATTAAAGGAAGCTCTATTAGATACAGGAGCAGATGATACAGTATT
AGAAGAAATGAATTTGCCAGGAAGATGGAAACCAAAAATGATAGGGGGAA
TTGGAGGTTTTATCAAAGTAAGACAGTATGATCAGATACTCATAGAAATC
TGCGGACATAAAGCTATAGGTACAGTATTAGTAGGACCTACACCTGTCAA
CATAATTGGAAGAAATCTGTTGACTCAGATTGGCTGCACTTTAAATTTTC
CCATTAGTCCTATTGAGACTGTACCAGTAAAATTAAAGCCAGGAATGGAT
GGCCCAAAAGTTAAACAATGGCCATTGACAGAAGAAAAAATAAAAGCATT
AGTAGAAATTTGTACAGAAATGGAAAAGGAAGGAAAAATTTCAAAAATTG
GGCCTGAAAATCCATACAATACTCCAGTATTTGCCATAAAGAAAAAAGAC
AGTACTAAATGGAGAAAATTAGTAGATTTCAGAGAACTTAATAAGAGAAC
TCAAGATTTCTGGGAAGTTCAATTAGGAATACCACATCCTGCAGGGTTAA
AACAGAAAAAATCAGTAACAGTACTGGATGTGGGCGATGCATATTTTTCA
GTTCCCTTAGATAAAGACTTCAGGAAGTATACTGCATTTACCATACCTAG
TATAAACAATGAGACACCAGGGATTAGATATCAGTACAATGTGCTTCCAC
AGGGATGGAAAGGATCACCAGCAATATTCCAGTGTAGCATGACAAAAATC
TTAGAGCCTTTTAGAAAACAAAATCCAGACATAGTCATCTATCAATACAT
GGATGATTTGTATGTAGGATCTGACTTAGAAATAGGGCAGCATAGAACAA
AAATAGAGGAACTGAGACAACATCTGTTGAGGTGGGGATTTACCACACCA
GACAAAAAACATCAGAAAGAACCTCCATTCCTTTGGATGGGTTATGAACT
CCATCCTGATAAATGGACAGTACAGCCTATAGTGCTGCCAGAAAAGGACA
GCTGGACTGTCAATGACATACAGAAATTAGTGGGAAAATTGAATTGGGCA
AGTCAGATTTATGCAGGGATTAAAGTAAGGCAATTATGTAAACTTCTTAG
GGGAACCAAAGCACTAACAGAAGTAGTACCACTAACAGAAGAAGCAGAGC
TAGAACTGGCAGAAAACAGGGAGATTCTAAAAGAACCGGTACATGGAGTG
TATTATGACCCATCAAAAGACTTAATAGCAGAAATACAGAAGCAGGGGCA
AGGCCAATGGACATATCAAATTTATCAAGAGCCATTTAAAAATCTGAAAA
CAGGAAAGTATGCAAGAATGAAGGGTGCCCACACTAATGATGTGAAACAA
TTAACAGAGGCAGTACAAAAAATAGCCACAGAAAGCATAGTAATATGGGG
AAAGACTCCTAAATTTAAATTACCCATACAAAAGGAAACATGGGAAGCAT
GGTGGACAGAGTATTGGCAAGCCACCTGGATTCCTGAGTGGGAGTTTGTC
AATACCCCTCCCTTAGTGAAGTTATGGTACCAGTTAGAGAAAGAACCCAT
AATAGGAGCAGAAACTTTCTATGTAGATGGGGCAGCCAATAGGGAAACTA
AATTAGGAAAAGCAGGATATGTAACTGACAGAGGAAGACAAAAAGTTGTC
CCCCTAACGGACACAACAAATCAGAAGACTGAGTTACAAGCAATTCATCT
AGCTTTGCAGGATTCGGGATTAGAAGTAAACATAGTGACAGACTCACAAT
ATGCATTGGGAATCATTCAAGCACAACCAGATAAGAGTGAATCAGAGTTA
GTCAGTCAAATAATAGAGCAGTTAATAAAAAAGGAAAAAGTCTACCTGGC
ATGGGTACCAGCACACAAAGGAATTGGAGGAAATGAACAAGTAGATAAAT
TGGTCAGTGCTGGAATCAGGAAAGTACTATTTTTAGATGGAATAGATAAG
GCCCAAGAAGAACATGAGAAATATCACAGTAATTGGAGAGCAATGGCTAG
TGATTTTAACCTACCACCTGTAGTAGCAAAAGAAATAGTAGCCAGCTGTG
ATAAATGTCAGCTAAAAGGGGAAGCCATGCATGGACAAGTAGACTGTAGC
CCAGGAATATGGCAGCTAGATTGTACACATTTAGAAGGAAAAGTTATCTT
GGTAGCAGTTCATGTAGCCAGTGGATATATAGAAGCAGAAGTAATTCCAG
CAGAGACAGGGCAAGAAACAGCATACTTCCTCTTAAAATTAGCAGGAAGA
TGGCCAGTAAAAACAGTACATACAGACAATGGCAGCAATTTCACCAGTAC
TACAGTTAAGGCCGCCTGTTGGTGGGCGGGGATCAAGCAGGAATTTGGCA
TTCCCTACAATCCCCAAAGTCAAGGAGTAATAGAATCTATGAATAAAGAA
TTAAAGAAAATTATAGGACAGGTAAGAGATCAGGCTGAACATCTTAAGAC
AGCAGTACAAATGGCAGTATTCATCCACAATTTTAAAAGAAAAGGGGGGA
TTGGGGGGTACAGTGCAGGGGAAAGAATAGTAGACATAATAGCAACAGAC
ATACAAACTAAAGAATTACAAAAACAAATTACAAAAATTCAAAATTTTCG
GGTTTATTACAGGGACAGCAGAGATCCAGTTTGGAAAGGACCAGCAAAGC
TCCTCTGGAAAGGTGAAGGGGCAGTAGTAATACAAGATAATAGTGACATA
AAAGTAGTGCCAAGAAGAAAAGCAAAGATCATCAGGGATTACGGAAAACA
GATGGCAGGTGACGATTGTGTGGCAAGTAGACAGGACGAGGATTAACACA
TGGAAAAGATTAGTAAAACACCATTAATATATTTCAAGGAAAGCTAAGGA
CTGGTTTTATAGACATCACTATGAAAGTACTAATCCAAAAATAAGTTCAG
AAGTACACATCCCACTAGGGGATGCTAAATTAGTAATAACAACATATTGG
GGTCTGCATACAGGAGAAAGAGACTGGCATTTGGGTCAGGGAGTCTCCAT
AGAATGGAGGAAAAAGAGATATAGCACACAAGTAGACCCTGACCTAGCAG
ACCAACTAATTCATCTGCACTATTTTGATTGTTTTTCAGAATCTGCTATA
AGAAATACCATATTAGGACGTATAGTTAGTTAAAGGTGTGAATATCAAGC
AGGACATAACAAGGTAGGATCTCTACAGTACTTGGCACTAGCAGCATTAA
TAAAACCAAAACAGATAAAGCCACCTTTGCCTAGTGTTAGGAAACTGACA
GAGGACAGCCCGAACAAGCCCCAGAAGACCAAGGGCCACAGAGGGAGCCA
TACAATGAATGGACACTAGAGCTTTTAGAGGAACTTAAGAGTGAAGCTGT
TAGACATTTTCCTAGGATATGGCTCCATAACTTAGGACAACATATCTATG
AAACTTACGGGGATACTTGGGCAGGAGTGGAATAAATAATAAGAATTCTG
CAACAACTGCTGTTTATCCATTTCAGAATTGGGTGTCGACATAGCAGAAT
AGGCGTTACTCGACAGAGGAGAGCAAGAAATGGAGCCAGTAGATCCTAGA
CTAGAGCCCTGGAAGCATCCAGGAAGTCAGCCTAAAACTGCTTGTACCAA
TTGCTATTGTAAAAAGTGTTGCTTTCATTGCCAAGTTTGTTTCATGACAA
AAGCCTTAGGCATCTCCTATGGCAGGAAGAAGCGGAGACAGCGACGAAGA
GCTCATCAGAACAGTCAGACTCATCAAGCTTCTCTATCAAAGCAGTAAGT
AGTACATGTACCCCAACCTATAATAGTAGCAATAGTAGCATTAGTAGTAG
CAATAATAATAGCAATAGTTGTGTGGTCCATAGTAATCATAGAATATAGG
AAAATATTAAGACAAAGAAAAATAGACTAATTAATTGATAGACTAATAGA
AAGAGCAGAAGACAGTGGCAATGAGAGTGAAGGAGAAGTATCAGCACTTG
TGGAGATGGGGGTGGAAATGGGGCACCATGCTCCTTGGGATATTGATGAT
CTGTAGTGCTACAGAAAAATTGTGGGTCACAGTCTATTATGGGGTACCTG
TGTGGAAGGAAGCAACCACCACTCTATTTTGTGCATCAGATGCTAAAGCA
TATGATACAGAGGTACATAATGTTTGGGCCACACATGCCTGTGTACCCAC
AGACCCCAACCCACAAGAAGTAGTATTGGTAAATGTGACAGAAAATTTTA
ACATGTGGAAAAATGACATGGTAGAACAGATGCATGAGGATATAATCAGT
TTATGGGATCAAAGCCTAAAGCCATGTGTAAAATTAACCCCACTCTGTGT
TAGTTTAAAGTGCACTGATTTGAAGAATGATACTAATACCAATAGTAGTA
GCGGGAGAATGATAATGGAGAAAGGAGAGATAAAAAACTGCTCTTTCAAT
ATCAGCACAAGCATAAGAGATAAGGTGCAGAAAGAATATGCATTCTTTTA
TAAACTTGATATAGTACCAATAGATAATACCAGCTATAGGTTGATAAGTT
GTAACACCTCAGTCATTACACAGGCCTGTCCAAAGGTATCCTTTGAGCCA
ATTCCCATACATTATTGTGCCCCGGCTGGTTTTGCGATTCTAAAATGTAA
TAATAAGACGTTCAATGGAACAGGACCATGTACAAATGTCAGCACAGTAC
AATGTACACATGGAATCAGGCCAGTAGTATCAACTCAACTGCTGTTAAAT
GGCAGTCTAGCAGAAGAAGATGTAGTAATTAGATCTGCCAATTTCACAGA
CAATGCTAAAACCATAATAGTACAGCTGAACACATCTGTAGAAATTAATT
GTACAAGACCCAACAACAATACAAGAAAAAGTATCCGTATCCAGAGGGGA
CCAGGGAGAGCATTTGTTACAATAGGAAAAATAGGAAATATGAGACAAGC
ACATTGTAACATTAGTAGAGCAAAATGGAATGCCACTTTAAAACAGATAG
CTAGCAAATTAAGAGAACAATTTGGAAATAATAAAACAATAATCTTTAAG
CAATCCTCAGGAGGGGACCCAGAAATTGTAACGCACAGTTTTAATTGTGG
AGGGGAATTTTTCTACTGTAATTCAACACAACTGTTTAATAGTACTTGGT
TTAATAGTACTTGGAGTACTGAAGGGTCAAATAACACTGAAGGAAGTGAC
ACAATCACACTCCCATGCAGAATAAAACAATTTATAAACATGTGGCAGGA
AGTAGGAAAAGCAATGTATGCCCCTCCCATCAGTGGACAAATTAGATGTT
CATCAAATATTACTGGGCTGCTATTAACAAGAGATGGTGGTAATAACAAC
AATGGGTCCGAGATCTTCAGACCTGGAGGAGGCGATATGAGGGACAATTG
GAGAAGTGAATTATATAAATATAAAGTAGTAAAAATTGAACCATTAGGAG
TAGCACCCACCAAGGCAAAGAGAAGAGTGGTGCAGAGAGAAAAAAGAGCA
GTGGGAATAGGAGCTTTGTTCCTTGGGTTCTTGGGAGCAGCAGGAAGCAC
TATGGGCGCAGCGTCAATGACGCTGACGGTACAGGCCAGACAATTATTGT
CTGATATAGTGCAGCAGCAGAACAATTTGCTGAGGGCTATTGAGGCGCAA
CAGCATCTGTTGCAACTCACAGTCTGGGGCATCAAACAGCTCCAGGCAAG
AATCCTGGCTGTGGAAAGATACCTAAAGGATCAACAGCTCCTGGGGATTT
GGGGTTGCTCTGGAAAACTCATTTGCACCACTGCTGTGCCTTGGAATGCT
AGTTGGAGTAATAAATCTCTGGAACAGATTTGGAATAACATGACCTGGAT
GGAGTGGGACAGAGAAATTAACAATTACACAAGCTTAATACACTCCTTAA
TTGAAGAATCGCAAAACCAGCAAGAAAAGAATGAACAAGAATTATTGGAA
TTAGATAAATGGGCAAGTTTGTGGAATTGGTTTAACATAACAAATTGGCT
GTGGTATATAAAATTATTCATAATGATAGTAGGAGGCTTGGTAGGTTTAA
GAATAGTTTTTGCTGTACTTTCTATAGTGAATAGAGTTAGGCAGGGATAT
TCACCATTATCGTTTCAGACCCACCTCCCAATCCCGAGGGGACCCGACAG
GCCCGAAGGAATAGAAGAAGAAGGTGGAGAGAGAGACAGAGACAGATCCA
TTCGATTAGTGAACGGATCCTTAGCACTTATCTGGGACGATCTGCGGAGC
CTGTGCCTCTTCAGCTACCACCGCTTGAGAGACTTACTCTTGATTGTAAC
GAGGATTGTGGAACTTCTGGGACGCAGGGGGTGGGAAGCCCTCAAATATT
GGTGGAATCTCCTACAGTATTGGAGTCAGGAACTAAAGAATAGTGCTGTT
AACTTGCTCAATGCCACAGCCATAGCAGTAGCTGAGGGGACAGATAGGGT
TATAGAAGTATTACAAGCAGCTTATAGAGCTATTCGCCACATACCTAGAA
GAATAAGACAGGGCTTGGAAAGGATTTTGCTATAAGCCCGGTGGCAAGTG
GTCAAAAAGTAGTGTGATTGGATGGCCTGCTGTAAGGGAAAGATAAAGAC
GAGCTGAGCCAGCAGCAGATGGGGTGGGAGCAGTATCTCGAGACCTAGAA
AAACATGGAGCAATCACAAGTAGCAATACAGCAGCTAACAATGCTGCTTG
TGCCTGGCTAGAAGCACAAGAGGAGGAAGAGGTGGGTTTTCCAGTCACAC
CTCAGGTACCTTTAAGACCAATGACTTACAAGGCAGCTGTAGATCTTAGC
CACTTTTTAAAAGAAAAGGGGGGACTGGAAGGGCTAATTCACTCCCAAAG
AAGACAAGATATCCTTGATCTGTGGATCTACCACACACAATAATACTTCC
CTGATTGGCAGAACTACACACCAGGGCCAGGGGTCAGATATCCACTGACC
TTTGGATGGTGCTACAAGCTAGTACCAGTTGAGCCAGATAAGGTAGAAGA
GGCCAATAAAGGAGAGAACACCAGCTTGTTACACCCTGTGAGCCTGCATG
GAATGGATGACCCTGAGAGAGAAGTGTTAGAGTGGAGGTTTGACAGCCGC
CTAGCATTTCATCACGTGGCCCGAGAGCTGCATCCGGAGTACTTCAAGAA
CTGCTGACATCGAGCTTGCTACAAGGGACTTTCCGCTGGGGACTTTCCAG
GGAGGCGTGGCCTGGGCGGGACTGGGGAGTGGCGAGCCCTCAGATGCTGC
ATATAAGCAGCTGCTTTTTGCCTGTACTGGGTCTCTCTGGTTAGACCAGA
TCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTG
TGACTCTGGTAACTAGAGATCCCTCAGACC

### REFERENCES

Cornetta et al. 2011 - “Replication-competent Lentivirus Analysis of Clinical Grade Vector Products”. Mol Ther. 2011 Mar; 19(3): 557-566.

Corre et al. 2016 - “RCL-Pooling Assay”: A Simplified Method for the Detection of Replication Competent Lentiviruses in Vector Batches Using Sequential Pooling″. Hum Gene Ther. Feb;27(2):202-10. doi: 10.1089/hum.2015.166.

Forestell, S., Dando, J., Böhnlein, E. and Rigg, R. (1996). Improved detection of replication-competent retrovirus. Journal of Virological Methods, 60(2), pp.171-178

Miskin, J., Chipchase, D., Rohll, J., Beard, G., Wardell, T., Angell, D., Roehl, H., Jolly, D., Kingsman, S. and Mitrophanous, K. (2005). A replication competent lentivirus (RCL) assay for equine infectious anaemia virus (EIAV)-based lentiviral vectors. Gene Therapy, 13(3), pp. 196-205.

Sastry, L., Xu, Y., Duffy, L., Koop, S., Jasti, A., Roehl, H., Jolly, D. and Cornetta, K. (2005). Product-Enhanced Reverse Transcriptase Assay for Replication-Competent Retrovirus and Lentivirus Detection. Human Gene Therapy, 16(10), pp.1227-1236

1. A method for detecting replication competent virus in a test sample comprising:
a) providing a plurality of individual cell culture aliquots each with maximum aqueous volume of less than 12 ml, wherein each aliquot comprises a portion of the sample and virus-permissive cells;
b) culturing the aliquots for at least nine days;
c) culturing the aliquots for at least a further six days, wherein the aliquots are passaged using a dilution factor of at least 2 at each passage; and
d) testing for the presence of replication competent virus.

2. The method of claim 1, wherein the virus is selected from the group consisting of: a retrovirus, an adenovirus, an adeno-associated virus, a herpes simplex virus and a vaccinia virus.

3. The method of any preceding claim, wherein the retrovirus is a lentivirus.

4. The method of any preceding claim, wherein the maximum aqueous volume of each individual cell culture aliquot in step a) is selected from: 11 ml, 10 ml, 5 ml or 3 ml.

5. The method of any preceding claim, wherein the total volume across all aliquots is reduced by at least 50% during step c).

6. The method of any preceding claim, wherein the test sample comprises viral particles or end of production cells.

7. The method of any preceding claim, wherein the virus-permissive cells are non-adherent.

8. The method of claim 7, wherein the virus-permissive cells are selected from:
a) immortalised T cell lines, optionally wherein the cells are selected from Jurkat, CEM-SS, PM1, Molt4, Molt4.8, SupT1, MT4 or C8166 cells; or
b) non-T cell lines, optionally wherein the cells are selected from HEK293 or 92BR cells.

9. The method of any preceding claim, wherein the total volume of the plurality of individual cell culture aliquots of step a) is at least about 115 ml.

10. The method of any preceding claim, wherein the initial seeding density of the plurality of individual cell culture aliquots in step a) is in the range of from about 1 × 10⁵ total cells/ml to about 1 × 10⁷ total cells/ml.

11. The method of claim 10, wherein the initial seeding density of the plurality of individual cell culture aliquots in step a) is in the range of from about 1 × 10⁶ total cells/ml to 1 × 10⁷ total cells/ml.

12. The method of any preceding claim, wherein step c) comprises culturing the aliquots for at least a further eight or nine days.

13. The method of any preceding claim, wherein each individual cell culture aliquot is within a cell culture vessel.

14. The method of claim 13, wherein the cell culture vessel is selected from a cell culture tube, a cell culture dish or a cell culture plate comprising a plurality of wells.

15. The method of claim 14, wherein the cell culture plate comprising a plurality of wells is selected from the group consisting of: a 4- well, 6- well, 8- well, 12- well, 24- well, 48- well, 96- well and a 384- well cell culture plate.

16. The method of any preceding claim, wherein the cell culture plate comprising a plurality of wells is a 12- well plate or a 24- well plate.

17. The method of any preceding claim, wherein the method is automated.

18. The method of any preceding claim, wherein the presence of replication competent virus is tested using PCR or ELISA.

19. The method of any preceding claim, wherein the presence of replication competent virus is tested using a reverse transcriptase assay.

20. The method of any preceding claim, wherein the method is for detecting replication competent lentivirus in the test sample, and the method is performed in parallel with a positive control sample comprising an attenuated replication competent lentivirus that has at least one accessory gene functionally mutated within its nucleotide sequence, wherein the at least one accessory gene is selected from: vif, vpr, vpx, vpu and nef.

21. The method of claim 20, wherein the method is for detecting replication competent HIV, SIV, SHIV in the test sample, or a variant thereof.

22. The method of claim 20 or 21, wherein the attenuated replication competent lentivirus has at least three of vif, vpr, vpx, vpu and nef functionally mutated.

23. The method of any of claims 20 to 22, wherein the attenuated replication competent virus comprises a nucleic acid sequence according to SEQ ID NO: 1.

24. The method of any preceding claim, wherein the method is for testing products for gene therapy.

25. A replication competent virus comprising a nucleic acid sequence according to SEQ ID NO: 1.
